(19) United States
(12) Patent Application Publication
(10) Pub. No.: US 2014/0287946 A1
(43) Pub. Date: Sep. 25, 2014

(54) NUCLEIC ACID CONTROL PANELS

(71) Applicant: IBIS BIOSCIENCES, INC., Carlsbad, CA (US)

(72) Inventor: Herbert A. Marble, Carlsbad, CA (US)

(73) Assignee: IBIS BIOSCIENCES, INC., CARLSBAD, CA (US)

(21) Appl. No.: 14/212,563

(22) Filed: Mar. 14, 2014

Related U.S. Application Data
(60) Provisional application No. 61/784,240, filed on Mar. 14, 2013.

(51) Int. Cl. C12Q 1/68

(52) U.S. Cl. CPC C12Q 1/6837
USPC 506/9; 506/16

(57) ABSTRACT

Provided herein is technology relating to detecting nucleic acids in a sample and particularly, but not exclusively, to systems and methods related to panels that are used to evaluate sequencing efficacy.

This application claims priority to U.S. provisional patent application Ser. No. 61/784,240, filed Mar. 14, 2013, which is incorporated herein by reference in its entirety.

### FIELD

Provided herein is technology relating to detecting nucleic acids in a sample and particularly, but not exclusively, to systems and methods related to panels that are used to evaluate nucleic acid assay efficacy.

### BACKGROUND

Mutations/variations in the human genome are involved in many diseases, ranging from monogenetic to multifactorial diseases, and acquired diseases such as cancer. Even the susceptibility to infectious diseases, and the response to pharmaceutical drugs, is affected by the composition of an individual's genome. Most genetic tests, which screen for such mutations/variations, require amplification of the DNA region under investigation. However, the size of the genomic DNA that can be amplified is rather limited, and there is often high signal noise. For example, the upper size limit of an amplified DNA fragment in a standard PCR reaction is about 2 Kb. This contrasts sharply with the total size of 3 billion nucleotides of which the human genome is composed. As more and more mutations/variations are found to be involved in disease, there is a need for robust assays in which different DNA regions, that harbor the different mutations/variations, are analyzed together. This may be achieved through multiplex amplification reactions.

The polymerase chain reaction (PCR) is a primer-directed in vitro reaction for the enzymatic amplification of a specific DNA fragment (Saiki, “Enzymatic Amplification of β-Actin Genomic Sequences and Restriction Site Analysis for Diagnosis of Sickle Cell Anemia”, Science 230: 1350-54 (1985)). PCR is generally considered one of the most sensitive and rapid method for detecting nucleic acids in a particular sample. PCR is well-known in the art and has been described in its basic forms, for example, in U.S. Pat. No. 4,683,195 to Mullis et al.; U.S. Pat. No. 4,683,202 to Mullis; U.S. Pat. No. 5,298,392 to Atlas et al.; and U.S. Pat. No. 5,437,990 to Burg et al. In typical PCR, an oligonucleotide primer pair for each target is provided wherein each primer pair includes a first nucleotide sequence complementary to a sequence flanking the 5′ end of the target nucleic acid sequence and a second nucleotide sequence complementary to a nucleotide sequence flanking the 3′ end of the target nucleic acid sequence. The nucleotide sequences of each oligonucleotide primer pair are typically specific to a particular target sequence or sequences to be detected and are designed not to cross-react with other non-target sequences.

The distinctive nature of the PCR process in producing a substantive quantity of DNA fragments of interest from an initial tiny amount of DNA sample has gained broad application in the fields of biomedical research and clinical diagnosis. For example, PCR has been widely used in the diagnosis of inherited disorders, the individualization of evidence samples in the forensics area, and the detection of bacterial and viral pathogens and potential bioterror agents. See, e.g., Erlich et al, “Recent Advances in the Polymerase Chain Reaction”, Science 252: 1643-51 (1991); Newton & Graham, PCR (Oxford, 1994); Sontakke, “Use of broad range16S rDNA PCR in clinical microbiology”, J Microbiol Methods 76: 217-25 (2009); Yang, “PCR-based diagnostics for infectious diseases: uses, limitations, and future applications in acute-care settings” Lancet Infect Dis 4: 337-48 (2004); Sninsky, “The polymerase chain reaction (PCR): a valuable method for retroviral detection”, Lymphology 23: 92-7 (1990); Fykse, “Detection of bioterror agents in air samples using real-time PCR”, J Appl Microbiol 105: 351-8 (2008).

For example, PCR has played a critical role in genotyping a vast number of genetic polymorphisms and individual variations which underlie the onset of many diseases, see, e.g., Shi, “Enabling Large-Scale Pharmacogenetic Studies by High-throughput Mutation Detection and Genotyping Technologies”, Clin Chem 47: 164-172 (2001), and forms part of standard laboratory tests to detect clinically relevant pathogens, see e.g., Riffelmann, “Nucleic Acid Amplification Tests for Diagnosis of Bordetella Infections”, J Clin Microbiol 43: 4925-4929 (2005).

Widespread applications notwithstanding, the use of PCR is quite often limited by the costs and time associated with designing and assembling PCR assays. At the initial stages, selecting a target typically involves bioinformatic analysis of known sequences to identify sequences specific for the required detection. Then, providing a template nucleic acid comprising the target for amplification involves choosing a molecular biological method appropriate for the source of the nucleic acid and applying it to the sample. For example, an environmental sample and a cultured bacterial isolate may involve using different protocols and reagents for preparing quality template. The PCR assay itself involves designing, selecting, and synthesizing oligonucleotide primers that will robustly and reproducibly amplify the target without, for example, amplifying non-target sequences or forming primer dimers and/or hairpins. Assembling a reaction requires providing target nucleic acid, nucleotides, primers, polymerase, buffers, and other components at the appropriate concentrations in a reaction vessel. Experiments can easily involve hundreds and thousands of individual reactions, each one requiring a precise measurement and delivery of these components into the appropriate reaction vessel. Performing the thermocycling of the PCR requires selecting and/or programming a series of temperature cycles that are tuned to the melting, annealing, and extension of the particular template(s) and primers in the reaction as well as the buffers, salts, and other components of the reaction. Finally, the resulting amplicon may require purification before detection and evaluation by a chosen detection method. For example, some applications may use a probe to determine if an amplicon is present, while some applications may use sequencing to provide more information about mutations, strain variation, etc., at single-nucleotide resolution. As each of these steps often requires validation, testing, and appropriate experimental controls, developing, performing, and evaluating the results of a PCR assay can be demanding on the attention and time of researchers already having limited resources. Moreover, user proficiency and knowledge of molecular biology, enzyme biochemistry, data analysis, etc., at an expert level is often required for the assay.

### SUMMARY

Provided herein is technology relating to detecting nucleic acids in a sample and particularly, but not exclusively, to systems and methods related to DNA panels that are used to evaluate nucleic acid assay efficacy. The technology finds use with a variety of nucleic acid assay platforms, including, but not limited to, sequencing (e.g., next-generation sequencing), digital PCR, other amplification reactions, and other nucleic acid detection and analysis modalities. The technology is illustrated herein, primarily via sequencing technologies. However, it should be understood that the technology finds use with other platforms.

In some embodiments, the invention described herein relates to an assay and analytical process control strategy that is applicable to next generation sequencing (NGS) based diagnostic assays as well as other nucleic acid technologies. The control strategy is platform agnostic and applies to all currently known sequencing methods including but not limited to sequencing by synthesis, sequencing by ligation, sequencing by hybridization, single molecule sequencing, real time sequencing, single molecule real time sequencing, sequencing by heat, and nanopore sequencing. In some embodiments, the assay control strategy described herein uses one or more synthetic panels of nucleic acids to directly measure the assay-specific analytical system performance characteristics in situ during a sequencing run. In some embodiments, the panel is specifically designed for the purpose of analytical process control for the detection of somatic DNA mutations. In some embodiments, the panel comprises a well-defined mixture of nucleic acid sequences whose composition challenges various analytical performance characteristics of sequencing methodology.

In some embodiments, the invention provides a system for monitoring the analytical performance of a sequencing reaction. In particular, the invention provides a direct mechanism for measuring in situ the inherent analytical sensitivity of a sequencing run. This information is useful for determining the limit of detection for somatic DNA mutations in a given sequencing run.

For example, in some embodiments, provided herein are methods for determining analytical sensitivity and/or specificity of a nucleic acid reaction (e.g., sequencing reaction, digital PCR, etc.) comprising one or more or all of the steps of: a) adding predetermined concentrations of a plurality of synthetic nucleic acids to a sample containing a target nucleic acid, wherein two or more different members of said plurality of synthetic nucleic acids differ from one another in concentration and sequence; b) subjecting the mixture from (a) to a nucleic acid amplification procedure in which the synthetic nucleic acids and the target nucleic acid are amplified; c) identifying the amplification products from (b) of the synthetic nucleic acids and target nucleic acid by, for example, conducting a nucleic acid sequencing reaction that generates a measurable signal; d) detecting the presence of or amount of target nucleic acid in the sample using the measurable signal from (c); and; (e) determining the analytical sensitivity of the detection in (d) by analyzing the measurable signal generated by the synthetic nucleic acids.

In some embodiments, the synthetic nucleic acids and the target nucleic acid differ by one or more single nucleotide polymorphisms. For example, in some embodiments, the synthetic nucleic acids differ from each other by the location of the single nucleotide polymorphism. In some embodiments, the synthetic nucleic acids (collectively) contain each possible variation of the base at the location of the single nucleotide polymorphism. In some embodiments, the synthetic nucleic acids differs from each other and/or the target nucleic acid by one or more of: homopolymer stretches of a single base repeated 2-25 times; short tandem repeats; GC content; AT content; telomeric, subtelomeric, or centromeric repeats; small nucleic acid deletions; copy number variations; and/or ribonucleic acid structures comprising one or more of the following: circles, pseudoknots, hairpins, self-complementary tails, single stranded pseudo circles, and transfer ribonucleic acid structures.

In some embodiments, the predetermined concentrations of synthetic nucleic acids differ from one another in molarity in ratios selected from the group consisting of: 1:1, 1:1.05, 1:10, 1:100, 1:1000, 1:10,000, 1:100,000, 1:1,000,000, and other ratios of the formula 1:10̂x where x is a positive number (e.g., integer). However, any other desired ratio may be used. In some embodiments, two or more of such different ratios (e.g., 3 or more, 4 or more, 5 or more, 6 or more, etc.; 3, 4, 5, 6, etc.) are represented by the different synthetic nucleic acids.

In some embodiments, provided herein are methods for detecting a mutant allele comprising one or more or all of the steps of: a) isolating nucleic acid from a sample comprising a target sequence having a mutation; b) adding to the isolated nucleic acid a plurality of different synthetic nucleic acids that contain synthetic versions of said target sequence such that the synthetic nucleic acids comprise a sequence 95-99.99% identical to the target sequence; c) amplifying the target sequence of the nucleic acid and amplifying the synthetic nucleic acids to generate amplification products (e.g., using amplification reagents); d) detecting the amplification products of the target nucleic acid (e.g., by detecting a measurable signal); e) detecting the amplification products of the synthetic nucleic acids (e.g., by detecting a measurable signal); and f) comparing the signal generated in (e) with the signal generated in (d).

In some embodiments, provided herein are methods for detecting a target nucleic acid in a background of non-target nucleic acid, wherein the target nucleic acid is in low concentration compared to the background non-target nucleic acids, comprising one or more or all of the steps of: a) obtaining a target nucleic acid from a sample containing a background nucleic acid; b) adding to the nucleic acid sequences in (a) a plurality of synthetic nucleic acids that, in some embodiments, differ from the target nucleic acid by one or more polymorphisms and that differ from each other by concentration; c) co-amplifying the synthetic nucleic acids and the target nucleic acid to generate amplification products; d) detecting the amplification products from (c) (e.g., using a detection method that generates a measurable signal); e) identifying the target nucleic acid based on the signal generated by the amplification of the nucleic acid sequences; and f) evaluating the accuracy of the identification in (e) by analyzing the signals generated by the amplified synthetic nucleic acid sequences.

In some embodiments, further provided herein are kits for carrying out any of the methods, the kits having one or more or all of the components necessary, useful, or sufficient to conducts the methods, including, as desired, positive and negative control reagents, containers, and software (e.g., data analysis software that calculates and reports assay results based on concentrations of reagents, measured signals, or other assay parameters). For example, in some embodiments, provided herein are kits for determining the specificity and/or sensitivity of a nucleic acid sequencing reaction comprising one or more or all of: a) a plurality of synthetic nucleic acids in predetermined concentrations that differ in sequence and concentration from each other and that differ in sequence from a target nucleic acid; b) nucleic acid amplification reagents comprising a plurality of primers that co-amplify said target nucleic acid and said plurality of synthetic nucleic acids; and c) nucleic acid sequencing reagents. In some embodiments, a positive control target nucleic acid sequence is provided.

In some embodiments, further provided herein are compositions (e.g., reaction mixtures) employed by the methods or using the kits. For example, in some embodiments, provided herein are compositions comprising: a plurality of synthetic nucleic acids in predetermined concentrations that differ in sequence and concentration from each other and that differ in sequence from a target nucleic acid; and nucleic acid amplification reagents comprising a plurality of primers that co-amplify said target nucleic acid and said plurality of synthetic nucleic acids. In some embodiments, provided herein are compositions comprising: a) amplicons generated from an amplification reaction employing the above composition; and b) sequencing reagents.

### BRIEF DESCRIPTION OF THE DRAWINGS

These and other features, aspects, and advantages of the present technology will become better understood with regard to the following drawings:

FIG. 1 is a drawing showing a template for NGS comprising a structure where the target sequence of interest is flanked by system-specific adaptor sequences.

FIG. 2 is a drawing showing an A-template control strand.

FIG. 3 is a drawing showing a panel constructed to represent each of the four nucleotides together on a control strand in aggregate.

FIG. 4 is a plot of mapped reads versus control panel oligonucleotide concentration for a somatic DNA control panel for SNP detection.

FIG. 5 is a plot of expected copy number versus measured copy number for a copy number variation control panel.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Rare allele (minor population) detection against a highly abundant and complex background is an important attribute for future Next Generation Sequencing (NGS) diagnostic sequencing applications related to clinical molecular diagnostic applications in oncology (e.g., somatic mutations, circulating tumor cells, and cell-free DNA), infectious disease (e.g., pathogen resistance profiling for viral, bacterial, and fungal agents), and genetics (e.g., fetal cells, DNA in maternal blood and bone marrow, and solid organ transplant rejection). For cancer, the ability to sensitively detect a mutant or variant somatic allele in an overwhelming excess of wild type germ line genotypes poses a formidable challenge. Likewise, discerning the presence of a minor population viral (or pathogen) species in a heterogeneous mixed sample (e.g., drug resistance typing, metagenomics, genotyping, population analysis, and multiple co-infections) remains an extremely difficult task that is often compounded by the inherent presence of a vast excess of host DNA.

Provided herein are systems, compositions, and methods for solving problems associated with such difficult tasks. For example, including a well-defined, synthetic DNA mutation control panel internally within a sequencing run or other nucleic acid assay (e.g., digital PCR, etc.) provides useful detail about the inherent analytical performance of the assay or system with respect to detecting a pre-calibrated set of standard reference DNA sequences precisely mixed in varying proportions. In some embodiments, a mutation panel is provided, comprised of a well-defined mixture of related DNA sequences differing from each other and, in some embodiments, from the analyte sequence, in some way at defined positions across the molecule, and present in different relative abundances. By including artificial nucleic acid sequences generally able to be co-amplified with the analyte nucleic acid in different proportions (e.g., 1:10, 1:100, 1:1000, 1:10,000, 1:100,000, and 1:1,000,000, etc.), one can measure the analytical sensitivity of the reaction against an internally added standard reference panel. In some embodiments, the panel represents each individual nucleotide base (A, C, G, and T) as an artificial SNP at different positions along a template molecule in a mixture of various proportions. Depending on the read length of the sequencing system (e.g., 25-50 bases, 50-75 bases, 100-200 bases, 200-500 bases, 500-1000 bases, etc.), mutations are placed strategically along a control template at the beginning, middle, and end to measure the efficiency of detection across an individual read. In some embodiments, a limited dilution panel is used for particular applications (e.g., 1:1.05, 1:10, 1:100, and 1:1000), while other applications may employ a broader dilution panel (e.g., 1:10 to 1:1,000,000). As such, the panel can be customized for specific applications and sequences.

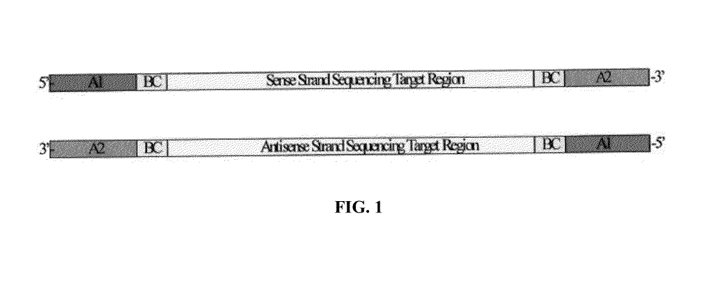

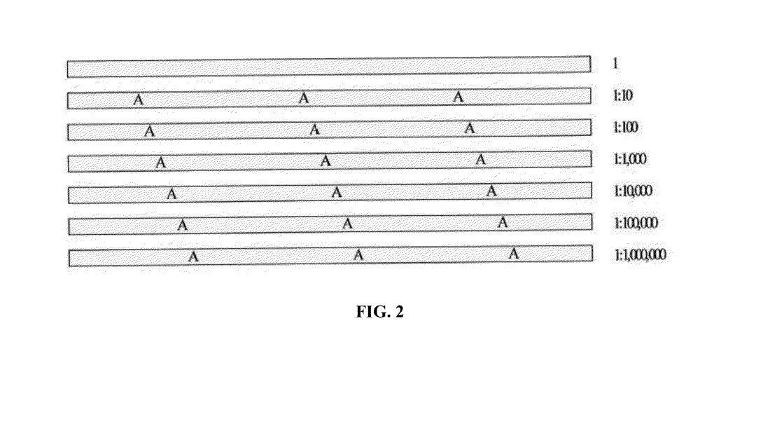

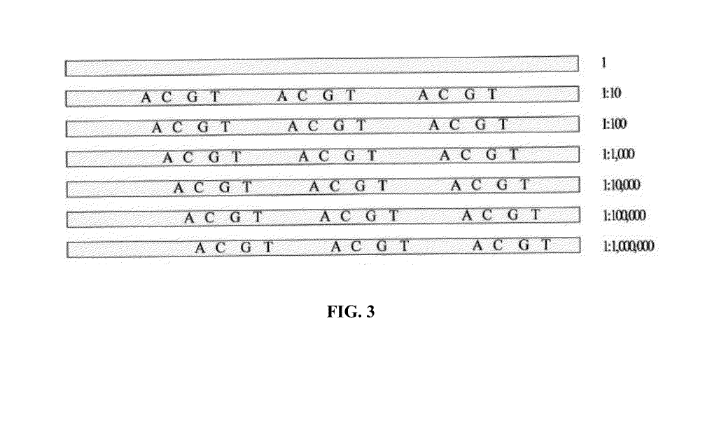

As depicted in FIG. 1, templates for NGS often involve a structure where the target sequence of interest is flanked by system-specific adaptor sequences, potentially with and without the inclusion of barcode sequences. Barcode sequences may be the preferred method for distinguishing artificial control sequences from samples as the unique sequence tags identifies the exogenously added reference samples. However, in some embodiments other methods such as the use of unique non-human DNA sequences (e.g., pumpkin DNA) may also be used to discriminate the control sequences from the sample. In some embodiments, both methods (barcodes and non-target (e.g., non-human) sequences) are employed to ensure distinction of control sequences from the desired (e.g., human) sample DNA. In some embodiments, the panel is constructed to individually represent each nucleotide on a separate DNA control strand (e.g., A, C, G, and T). The A-template control strand is shown in FIG. 2. In other embodiments, the panel is constructed to represent each of the four nucleotides together on a control strand in aggregate as shown in FIG. 3. For the latter, the individual bases are separated and spaced along the sequence at defined positions. Each region (e.g., beginning, middle, and end) may be further defined by a unique sequence orientation (e.g., ACGT, GATC, and TCAG) to unambiguously identify the three SNP clusters depicted along the control targets.

In some embodiments, the controls are prepared separately as individual libraries and added directly to the sample prior to clonal amplification (if amplification is employed) and sequencing. In other embodiments, the controls are added during the library preparation steps. Addition prior to clonal amplification and sequencing ensures that each of the components of the control panel is present precisely in the desired relative abundance. This eliminates inefficiencies and imbalances imparted during the preceding sample and library preparation steps. In some embodiments, the total amount of control material added to the sample is empirically determined for each system based on throughput and available real estate coverage and may vary across different platforms and for different applications.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in some embodiments” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products (“amplicons”) are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

The term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N⁶-methyladenosine, aziridinylcytosine, pseudoisocytosine, 5-(carboxyhydroxyl-methyl)-uracil, 5-fluorouracil, 5-bromouracil, 5-carboxymethylaminomethyl-2-thiouracil, 5-carboxymethyl-aminomethyluracil, dihydrouracil, inosine, N⁶-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N⁶-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

It is well known that DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

The term “communicate” refers to the direct or indirect transfer or transmission, and/or the capability of directly or indirectly transferring or transmitting, something at least from one thing to another thing. Objects “fluidly communicate” with one another when fluidic material is, or is capable of being, transferred from one object to another. Objects are in “thermal communication” with one another when thermal energy is or can be transferred from one object to another. Objects are in “magnetic communication” with one another when one object exerts or can exert a magnetic field of sufficient strength on another object to effect a change (e.g., a change in position or other movement) in the other object. Objects are in “sensory communication” when a characteristic or property of one object is or can be sensed, perceived, or otherwise detected by another object. It is to be noted that there may be overlap among the various exemplary types of communication referred to above.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

“Nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, Fla.), all herein incorporated by reference in their entireties.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981)J Am Chem Soc 103:3185-3191; automated synthesis methods; or the solid support method of U.S. Pat. No. 4,458,066, or other methods known to those skilled in the art. All of these documents are incorporated by reference.

A “polymerase” is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus DNA polymerase, Tth DNA polymerase, Vent DNA polymerase (New England Biolabs), Deep Vent DNA polymerase (New England Biolabs), Bst DNA Polymerase Large Fragment, Stoeffel Fragment, 9°N DNA Polymerase, Pfu DNA Polymerase, Tfl DNA Polymerase, RepliPHI Phi29 Polymerase, Tli DNA polymerase, eukaryotic DNA polymerase beta, telomerase, Therminator polymerase (New England Biolabs), KOD HiFi DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and 6,395,524 (incorporated by reference). These polymerases include wild-type, mutant isoforms, and genetically engineered variants.

As used herein a “sample” refers to anything capable of being analyzed by the methods and systems provided herein. In some embodiments, the sample comprises or is suspected to comprise one or more nucleic acids capable of analysis by the methods. In certain embodiments, for example, the samples comprise nucleic acids (e.g., DNA, RNA, cDNAs, etc.) from one or more organisms, tissues, cells, or environmental samples. Samples can include, for example, blood, semen, saliva, urine, feces, rectal swabs, and the like (e.g., whole blood, lymphatic fluid, serum, plasma, buccal, sweat, tears, saliva, sputum, hair, skin, biopsy, cerebrospinal fluid (CSF), amniotic fluid, seminal fluid, vaginal excretions, serous, fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone, marrow, fine needle, etc.) or washes (e.g., oral, nasopharangeal, bronchial, bronchialalveolar, optic, rectal, intestinal, vaginal, epidermal, etc.) and/or other specimens). In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “solid support” is a solid material having a surface for attachment of molecules, compounds, cells, or other entities. The surface of a solid support can be flat or not flat. A solid support can be porous or non-porous. A solid support can be a chip or array that comprises a surface, and that may comprise glass, silicon, nylon, polymers, plastics, ceramics, or metals. A solid support can also be a membrane, such as a nylon, nitrocellulose, or polymeric membrane, or a plate or dish and can be comprised of glass, ceramics, metals, or plastics, such as, for example, polystyrene, polypropylene, polycarbonate, or polyallomer. A solid support can also be a bead, resin or particle of any shape. Such particles or beads can be comprised of any suitable material, such as glass or ceramics, and/or one or more polymers, such as, for example, nylon, polytetrafluoroethylene, TEFLON, polystyrene, polyacrylamide, sepaharose, agarose, cellulose, cellulose derivatives, or dextran, and/or can comprise metals, particularly paramagnetic metals, such as iron.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

A “system” denotes a set of components, real or abstract, comprising a whole where each component interacts with or is related to at least one other component within the whole. For example, a “system” in the context of analytical instrumentation refers a group of objects and/or devices that form a network for performing a desired objective.

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “target” refers to a nucleic acid sequence or structure to be detected or characterized.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, modular random access vessel, etc.).

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form.

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

### Embodiments of the Technology

Embodiments of the present invention provide systems, compositions, and methods for therapeutic, clinical, research, and industrial use. Exemplary applications are discussed herein, particularly focused on sequencing reactions. Additional uses will be apparent to one of ordinary skill in the art upon reading this disclosure.

In some embodiments, the invention is useful for determining the limit of detection of minor population rare allele(s) against a highly abundant and complex background of DNA (e.g., host and pathogen DNA). Although the disclosure herein refers to certain illustrated embodiments, it is to be understood that these embodiments are presented by way of example and not by way of limitation.

A. Somatic Mutation Control Panel

Including a control panel internally within an assay provides useful detail about the inherent analytical performance of the assay or system with respect to detecting a pre-calibrated set of standard reference sequences mixed in varying proportions. In some embodiments, a Somatic DNA Mutation Panel is provided comprised of a mixture of related nucleic acid sequences (e.g., DNA) differing by single nucleotides (e.g., artificial SNPs) at defined positions across the molecule, and present in different relative abundances. By including artificial nucleic acid sequences in different proportions (e.g., 1:10, 1:100, 1:1000, 1:10,000, 1:100,000, and 1:1,000,000), one can measure the analytical sensitivity of the reaction against an internally added standard reference panel. In some embodiments, the panel represents each individual nucleotide base (A, C, G, and T) as an artificial SNP at different positions along a template molecule in a mixture of various proportions. Depending on the read length of the sequencing system (e.g., 25-50 bases, 50-75 bases, 100-200 bases, 200-500 bases, 500-1000 bases, etc.), artificial SNPs can be placed strategically along a control template at the beginning, middle, and end to measure the efficiency of detection across an individual read. It may be desirable to use a limited dilution panel for some applications (e.g., 1:10, 1:100, and 1:1000). A broader dilution panel (e.g., 1:10 to 1:1,000,000) can be used, for example, when or where increased NGS real-estate improvements exist and/or assay sensitivity requirements require or benefit from such. As such, the panel can be customized for specific applications and sequences. In some embodiments the synthetic nucleic acid sequences are co-amplified with the analyte nucleic acid sequences.

Such panels find broad use, including in oncology assays, including multiplex assays with markers that may reside in a sample at low abundance relative to wild-type sequences or background nucleic acid.

B. DNA Control Panel with Homopolymer Stretches

In some embodiments, a DNA Control Panel with Homopolymer Stretches is provided, which is comprised of a mixture of related DNA sequences differing by regions containing homopolyer stretches of one or more base (e.g., A, C, G, or T in repeats of 2 to 25 bases) at defined positions across the molecule, and present in different relative abundances.

Such panels find broad use, including in viral genome assays (e.g., HIV), for assisting in the selection of therapeutic responses and monitoring therapeutic efficacy.

C. DNA Control Panel for Short Tandem Repeats

In some embodiments, a DNA Control Panel for Short Tandem Repeats is provided, which is comprised of a mixture of related DNA sequences differing by short tandem repeats (STRs) at defined positions across the molecule, and present in different relative abundances. All types of STRs are contemplated, including STRs of all possible sequence contexts in doublets (AG, AC, AT, and the like), triplets (AGA, AGC, ACA, and the like), and quadruplets (AGCA, AGGT, and the like). STRs of any length are contemplated (e.g., doublet, triplet, quadruplet, and so on up to dodecamer repeats and beyond).

Such panels find broad use, including in genetic assays for fragile X syndrome, cystic fibrosis, and the like.

D. DNA Control Panel for GC Content

In some embodiments, a DNA Control Panel for GC Content is provided, which is comprised of a mixture of related DNA sequences differing by GC content at defined positions across the molecule, and present in different relative abundances. In some embodiments, the DNA Control Panel for GC Content contains DNA sequences that co-amplify with an analyte DNA sequence, and the primary difference between the synthetic and analyte DNA sequences are GC content (e.g., 50%, 60%, 70%, 80%, 90% GC content, and the like).

Such panels find broad use, including in infectious disease assays for bacterial genome sequencing and metagenomic analyses.

E. DNA Control Panel for AT Content

In some embodiments, a DNA Control Panel for AT Content is provided, which is comprised of a mixture of related DNA sequences differing by AT content at defined positions across the molecule, and present in different relative abundances. In some embodiments, the DNA Control Panel for AT Content contains DNA sequences that co-amplify with an analyte DNA sequence, and the primary difference between the synthetic and analyte DNA sequences are AT content (e.g., 50%, 60%, 70%, 80%, 90% AT content, and the like).

Such panels find broad use, including in infectious disease assays for bacterial genome sequencing and metagenomic analyses.

F. DNA Control Panel for Telomeric Repeats

In some embodiments, a DNA Control Panel for Telomeric Repeats is provided, which is comprised of a mixture of related DNA sequences differing by repeats commonly associated with telomeres (telomeric repeats). For example, telomeric repeats comprising TTAGGG, (TTAGGG)2, (TTAGGG)n, CCCTAA, (CCCTAA)2, (CCCTAA)n, and others are located at defined positions across the synthetic molecule, and present in different relative abundances. All types and sizes of telomeric repeats are contemplated.

Such panels find broad use, including in genetics and oncology assays for measuring the extent of telomere repeat sequences and chromosome integrity (telomere length & shortening).

G. DNA Control Panel for Subtelomeric Repeats

In some embodiments, a DNA Control Panel for Subtelomeric Repeats is provided, which is comprised of a well-defined mixture of related DNA sequences differing by repeats commonly associated with subtelomeres (subtelomeric repeats). For example, subtelomeric repeats comprising TTAGGG, (TTAGGG)2, (TTAGGG)n, and others are located at defined positions across the synthetic molecule and present in different relative abundances. All types and sizes of subtelomeric repeats are contemplated.

Such panels find broad use, including in genetics and oncology assays for measuring the extent of subtelomere repeat sequences and chromosome integrity (subtelomere repeat length).

H. DNA Control Panel for Centromeric Repeats

In some embodiments, a DNA Control Panel for Centromeric Repeats is provided, which is comprised of a well-defined mixture of related DNA sequences differing by repeats commonly associated with centromeres (centromeric repeats). For example, centromeric repeats (TGGAA)ₙ comprising regions repeats of variable length of nucleic acid sequences associated with the centromere are located at defined positions across the synthetic molecule, and present in different relative abundances. All types and sizes of centromeric repeats are contemplated.

Such panels find broad use, including in genetics and oncology assays for measuring the extent of centromere repeat sequences and chromosome integrity (centromere repeat length).

I. RNA Structural Controls for Nanopore RNA Sequencing Applications

In some embodiments, an RNA Control Panel for Nanopore RNA Sequencing Applications is provided, which is comprised of a well-defined mixture of related RNA sequences differing by regions useful for RNA sequencing applications. For example, circles, pseudoknots, hairpins, self-complementary tails, single-stranded pseudo circles, tRNA-like structures and the like are located at defined positions across the synthetic molecule and present in different relative abundances.

Such panels find broad use, including structural controls for nanopore sequencing applications.

J. Small DNA Deletion Detection Controls

In some embodiments, a Small DNA Deletion Detection Control panel is provided, which is comprised of a well-defined mixture of related DNA sequences differing by specified deletions of 1-100 bases or more. For example, synthetic nucleic acid sequences differ from analyte nucleic acid sequences by only deleted base pairs located at defined positions across the synthetic molecule and present in different relative abundances. All types and sizes of nucleic acid deletions are contemplated. Such controls find particular use for assays assessing a variety of related deletions differing in size or sequence (e.g., epidermal growth factor receptor (EGFR) exon 19 deletions for assessment of cancer risk and/or selection of therapies).

K. DNA Copy Number Variation Controls

In some embodiments, a DNA Copy Number Variation (CNV) detection control panel is provided, which is comprised of a well-defined mixture of related DNA sequences differing by a 5′-Tag sequence useful for CNV quantitation and digital molecular counting applications. For example, synthetic nucleic acids mixed at pre-defined molar ratios (stoichiometric concentrations) and containing differing 5′-Tag sequences are used as positive internal controls for measuring CNVs. Such controls find particular use for CNV detection and digital molecular counting applications (e.g. gene amplifications, aneuploidy analysis, and fetal aneuploidy detection by non-invasive prenatal testing).

L. Synthesis and Construction of Nucleic Acids

The technology provided herein is not limited by the methods, processes, or technologies used to construct and/or synthesize the nucleic acids in the control panels described herein. Further, the technology encompasses control panels comprising single-stranded nucleic acids and/or control panels comprising double-stranded nucleic acids. In some embodiments, the single stranded and/or the double stranded nucleic acids comprise one or more adaptor sequences (e.g., comprising, in some embodiments, a barcode nucleic acid sequence) at the 5′ end and/or at the 3′ end.

For example, in some embodiments a control panel oligonucleotide is synthesized as a single-stranded nucleic acid. In some embodiments, an adaptor sequence (e.g., a single stranded adaptor sequence) is added (e.g., ligated) to the 5′ end of the oligonucleotide and/or an adaptor sequence (e.g., a single stranded adaptor sequence) is added (e.g., ligated) to the 3′ end of the oligonucleotide. Then, in some embodiments, nucleic acid synthesis (e.g., a polymerase chain reaction) is used to generate a double stranded control panel oligonucleotide comprising, in some embodiments, an adaptor sequence at the 5′ end and/or at the 3′ end.

In some embodiments, a single stranded oligonucleotide is synthesized comprising the control panel oligonucleotide and an adaptor sequence at the 5′ end and/or at the 3′ end. Then, in some embodiments, nucleic acid synthesis (e.g., a polymerase chain reaction) is used to generate a double stranded control panel oligonucleotide comprising an adaptor sequence at the 5′ end and/or at the 3′ end. In some embodiments, a single stranded oligonucleotide is synthesized comprising the control panel oligonucleotide and an adaptor sequence at the 5′ end and/or at the 3′ end, a complementary single stranded oligonucleotide is synthesized comprising a reverse complement of the control panel oligonucleotide and an adaptor sequence at the 5′ end and/or at the 3′ end, and the two oligonucleotides are hybridized (e.g., annealed) to one another to provide a double stranded nucleic acid comprising the control panel oligonucleotide and an adaptor sequence at the 5′ end and/or at the 3′ end.

In some embodiments, a single stranded oligonucleotide is synthesized comprising the control panel oligonucleotide, a complementary single stranded oligonucleotide is synthesized comprising a reverse complement of the control panel oligonucleotide, and the two oligonucleotides are hybridized (e.g., annealed) to provide a double stranded nucleic acid comprising the control panel oligonucleotide. Then, an adaptor sequence (e.g., a double stranded adaptor sequence) is added (e.g., ligated) to the 5′ end of the oligonucleotide and/or an adaptor sequence (e.g., a double stranded adaptor sequence) is added (e.g., ligated) to the 3′ end of the oligonucleotide to provide a double stranded nucleic acid comprising the control panel oligonucleotide and an adaptor sequence at the 5′ end and/or at the 3′ end.

In some embodiments, a double stranded nucleic acid comprising the control panel oligonucleotide and/or a double stranded nucleic acid comprising the control panel oligonucleotide and an adaptor sequence at the 5′ end and/or at the 3′ end is produced by amplification (e.g., PCR) from a plasmid, BAC, or other template comprising a nucleic acid comprising the control panel oligonucleotide and/or comprising a double stranded nucleic acid comprising the control panel oligonucleotide and an adaptor sequence at the 5′ end and/or at the 3′ end. In some embodiments, a double stranded nucleic acid comprising the control panel oligonucleotide and/or a double stranded nucleic acid comprising the control panel oligonucleotide and an adaptor sequence at the 5′ end and/or at the 3′ end is produced by restriction digest of a nucleic acid (e.g., a plasmid, a BAC, or other nucleic acid) comprising a nucleic acid comprising the control panel oligonucleotide and/or comprising a double stranded nucleic acid comprising the control panel oligonucleotide and an adaptor sequence at the 5′ end and/or at the 3′ end (e.g., and isolating the restriction fragment comprising the control panel oligonucleotide and/or a double stranded nucleic acid comprising the control panel oligonucleotide and an adaptor sequence at the 5′ end and/or at the 3′ end).

Embodiments provide that nucleic acids are synthesized using phosphoramidite methods (e.g., accompanied by linking to a solid support) known in the art and/or by any extant or yet-developed technology for synthesizing nucleic acids. In some embodiments, nucleic acids are produced by connecting (e.g., ligating) one or more nucleic acids together. In such embodiments, the one or more nucleic acids are independently (e.g., individually) provided by synthesis, restriction, hybridization, etc.

Further, the technology is not limited to the particular sequences (e.g., the nucleic acids and nucleotide sequences provided herein, e.g., as “Oligo” and “Seq ID No”) described herein. The specific nucleic acids and nucleotide sequences are exemplary and do not limit the technology. The technology described herein encompasses embodiments that are practiced using nucleic acids having other designs and/or comprising other nucleotide sequences that satisfy the same purposes for which the oligonucleotide control panels are described and applied.

M. Sequencing Methods

In some embodiments, the disclosure relates generally to methods, compositions, systems, apparatuses and kits for obtaining sequence information from at least a portion of a nucleic acid. In some embodiments, obtaining sequencing information can include sequencing by label-free or ion based sequencing methods. In some embodiments, obtaining sequencing information can include labeled or optically detectable based sequencing methods such as fluorescence or bioluminescence. In some embodiments, obtaining sequencing information can include determining the identity of an incorporated nucleotide by monitoring sequencing reaction byproducts released during nucleotide incorporation. In some embodiments, the sequencing reaction byproducts released during nucleotide incorporation can include hydrogen ions, inorganic pyrophosphate or inorganic phosphate.

In some embodiments, the disclosure relates generally to methods, compositions, systems, apparatuses and kits for obtaining sequence information from a nucleic acid via paired-end sequencing. In some embodiments, the nucleic acid can include a DNA, RNA, cDNA, mRNA, microRNA, or DNA/RNA hybrid. In some embodiments, the nucleic acid can be a target-specific nucleic acid associated with genotyping, such as a nucleic acid containing a single nucleotide polymorphism or a short tandem repeat. In some embodiments, the nucleic acid can be a target-specific nucleic acid associated with one or more medically relevant or medically actionable mutations, such as mutations associated with cancer or inherited disease. In some embodiments, the nucleic acid can be derived from a mammal such as a human.

In some embodiments, the method (and related compositions, systems, apparatuses and kits using the disclosed methods) can include obtaining sequencing information from a nucleic acid linked to a support. Optionally, the support can include any suitable support such as, but not limited to a bead, particle, microparticle, microsphere, slide, flowcell or reaction chamber. In some embodiments, the support can include a solid support. In some embodiments, the support can include a planar support such as a flowcell or slide. In some embodiments, the support can include an Ion Sphere Particle (ISP). In some embodiments, the nucleic acid includes a template strand. In some embodiments, the template strand can further include one or more adaptors. In some embodiments, the one or more adaptors can optionally include a barcode or tagging sequence. In some embodiments, a template strand including an adaptor can further include one or more nucleotide residues that are resistant to a degrading agent. In some embodiments, an adaptor can include one or more phosphorothioate or 2-O-Methyl RNA (2′ OMe) nucleotides. In some embodiments, the template strand can be linked to a support through the 5′ end of the template strand.

In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology finds use in nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the technology finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

The technology finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectable fluorescence resonance energy transfer (FRET) upon nucleotide addition.

### EXAMPLES

These examples describe exemplary DNA next-generation sequencing control panels for a variety of different potential target sequence types. In some embodiments, DNA control panels are added directly to (spiked in) the final NGS library preparation (DNA sequencing sample) prior to the system loading and clonal amplification steps (if necessary) by either 1) bridge PCR (Illumina GAIIx, HiSeq 2000, HiSeq 2500/1500, and MiSeq; Qiagen/IBS GeneRead nanoball chemistry) 2) emulsion PCR (Roche 454, Life Technologies SOLiD, Life Technologies Ion Torrent PGM & Proton, and GnuBio sequencing by hybridization platform), 3) template loading for single molecule sequencing systems (PacBio RS SMRT Cells with SMRT Bell libraries; Helicos HelioScope, Life Technologies VisiGen/StarLight), and 4) template loading for nanopore sequencing systems (Oxford Nanopore GridION and MinION, NobleGen, Genia, and others). Pre-quantitated synthetic DNA control panels (containing NGS platform-specific adaptor/primer sequences and at equimolar concentration with the DNA sample library) are introduced to the pre-quantitated NGS library sample by diluting/mixing at any desired and pre-defined volume (molar) ratio (such as, 1:1, 1:5, 1:10, 1:20, 1:50 1:100, 1:200, 1:500, 1:1,000, 1:10,000 volume, or as otherwise practical/desirable). Synthetic DNA control panels are treated identically as DNA sample NGS libraries for the specific NGS platform employed (e.g. Illumina, SOLiD, Ion Torrent, Roche, Pacific Biosciences, Qiagen, Oxford Nanopore, GnuBio, and others); in terms of solvent/diluent, buffers (pH), ionic strength (salt composition), molar concentration (measured by the method specified by the NGS platform for library quantitation, and at equimolar concentration with the actual NGS library sample). Synthetic DNA control panels are designed to include any requisite NGS adaptor or PCR primer sequences (with or without sample barcoding/indexes) flanking the control panel template sequence for the desired application (e.g. Somatic Mutation panels, Homopolymer panels, % GC panels, % AT panels, Short Tandem Repeat Sequence panels, Deletion panels, or any multiple combination thereof). Sequencing barcodes, molecular sequencing tags, unique identifiers, and indexes can also be included in the synthetic oligonucleotide design comprising the flanking regions for the DNA control panels (as appropriate for the NGS platform employed).

Alternatively, the DNA control panels are added directly to (spiked in) the input DNA sample (DNA sequencing sample) prior to NGS library construction and preparation (employing methods appropriate for the chosen NGS platform; e.g. Illumina, SOLiD, Ion Torrent, Roche, Pacific Biosciences, Qiagen, Oxford Nanopore, GnuBio, and others). This approach may be less preferable since the representation, composition, relative abundances, fidelity and integrity of the DNA control panel cannot be necessarily ensured throughout the series of platform-specific molecular biology steps involved in NGS library construction and preparation (converting an input DNA sample into an NGS library for sequencing on a specific NGS instrument platform). Regardless of these limitations, this method may be desired for alternate design or performance considerations. In this case, pre-quantitated synthetic DNA control panels are introduced to the pre-quantitated input DNA specimen by diluting and/or mixing at any desired and pre-defined volume (molar) ratio (such as, 1:1, 1:5, 1:10, 1:20, 1:50 1:100, 1:200, 1:500, 1:1,000, 1:10,000 volume; or as otherwise practical/desirable). The “spiked-in sample” (containing the desired DNA control panel introduced at the desired level) is then used directly as the input, starting DNA material for platform-specific NGS library construction and preparation.

In some embodiments, DNA control panels are comprised of human and/or non-human DNA sequence elements. In most cases, it is preferable to utilize a foreign, non-human DNA sequence that is either synthetically derived or uniquely expressed in another species (e.g. pumpkin DNA sequence elements). In other cases, such as deletions (indels), it may be preferable to include a synthetic DNA template that mimics and spans the actual deletion breakpoint boundary; in order to demonstrate the ability to detect the specific deletion or complex indel event. In such cases, it is important to maintain and distinguish the identity of the control sequence template (DNA control panel) from the actual test sample. This can be accomplished by employing sequencing barcodes, molecular sequencing tags, unique identifiers, and indexes; and/or alternatively by employing unique sequence keys & identifiers along the template spine and immediately flanking the artificial human deletion breakpoint boundary sequence.

Several examples of different control panels for different sequence analysis types are provided below. While not fully shown, in some embodiments, the sequences have the structure (barcode sequences are optional and can be placed symmetrically or asymmetrically flanking the control panel sequence):

### 5′-NGS Platform-Specific Adaptors/Primers-Platform-Specific Barcode-Control Panel Sequence-Platform Specific Barcode-NGS Platform Specific Adaptors/Primers-3′

### Example 1

### Exemplary Control Sequences

### Exemplary DNA Somatic Mutation Control Panel Sequence (Flanked by NGS Platform-Specific Adaptors & Barcodes; not Shown):

Somatic DNA mutation panels have practical utility for directly (in situ) and empirically measuring the effective sensitivity and limit of detection of the NGS system for measuring nucleotide substitution events (SNPs). Somatic DNA mutation panels can be added to DNA purified from patient tumor samples by the methods described above (clinical and/or research specimens derived from individuals with hematological disorders, solid tumors, and/or malignancies), in order to measure the analytical performance characteristics (e.g. sensitivity, linearity, upper & lower limit of detection, upper and lower limit of quantitation) of an NGS cancer/oncology sequencing panel (organ-specific cancer, pan-cancer, cancer of unknown origin). Several examples of somatic DNA mutation panels are detailed below.

1) Random Synthetic Sequence (100-mer)

Base Sequence (artificial wildtype)

(SEQ ID NO: 1)

5′-ACGTTGCATA CTGACCTAGG TAAGCGTTGC GAATCTGGAT
ATGCTTAACC CATGGATCAA TTCGACGCGG GTTACGCCTA
AATTGGCCAG CGTTAGCTAA-3′
1:10 SNP in Base Sequence Background
(artificial wildtype)

(SEQ ID NO: 2)

5′-ACGTTGCATG CTGACCTAGG TAAGCGTTGC GAATCTGGAT
CTGCTTAACC CATGGATCAC TTCGACGCGG GTTACGCCTA
AATTGGCCTG CGTTAGCTAA-3′
1:100 SNP in Base Sequence Background
(artificial wildtype)

(SEQ ID NO: 3)

5′-ACGTTGCATC CTGACCTAGG TAAGCGTTGC GAATCTGGAT
TTGCTTAACC CATGGATCAT TTCGACGCGG GTTACGCCTA
AATTGGCCCG CGTTAGCTAA-3′
1:1,000 SNP in Base Sequence Background
(artificial wildtype)

(SEQ ID NO: 4)

5′-ACGTTGCATT CTGACCTAGG TAAGCGTTGC GAATCTGGAT
GTGCTTAACC CATGGATCAG TTCGACGCGG GTTACGCCTA
AATTGGCCGG CGTTAGCTAA-3′
1:10,000 SNP (artificial wildtype)

(SEQ ID NO: 5)

5′-ACGTTGCATA CAGACCTAGG TAAGCGTTGC GAATCTGGAC
ATGCTTAACC CATGGATCAA GTCGACGCGG GTTACGCCTA
AATTGGCCAG TGTTAGCTAA-3′
1:100,000 SNP in Base Sequence Background
(artificial wildtype)

(SEQ ID NO: 6)

5′-ACGTTGCATA CCGACCTAGG TAAGCGTTGC GAATCTGGAG
ATGCTTAACC CATGGATCAA CTCGACGCGG GTTACGCCTA
AATTGGCCAG TGTTAGCTAA-3′

### Exemplary DNA Homopolymer Control Panel Sequence (Flanked by NGS Platform-Specific Adaptors & Barcodes; not Shown):

1) Random Synthetic Sequence (100-mer)

Base Sequence (artificial wildtype)

(SEQ ID NO: 7)

5′-ACGTTGCATA CTGACCTAGG TAAGCGTTGC GAATCTGGAT
ATGCTTAACC CATGGATCAA TTCGACGCGG GTTACGCCTA
AATTGGCCAG CGTTAGCTAA-3′
N = 2 Homopolymer in Base Sequence Background
(near artificial wildtype) (100-mer)

(SEQ ID NO: 8)

5′-ACGTTGCATT CTGACCTAGG TAAGCGTTGC GAATCTGGAT
ATGCCTAACC CATGGATCAA TTCGACGCCG GTTACGCCTA
AATTGGCCAG CGTTAGCTAA-3′
N = 3 Homopolymer in Base Sequence Background
(near artificial wildtype) (100-mer)

(SEQ ID NO: 9)

5′-ACGTTGCTTT CTGACCTAGG GAAGCGTTGC GAAACTGGAT
ATGCCCAACC CATGGATCAA ATCGACGCCC GTTACGCCTA
AATTGGGCAG CGTTTGCTAA-3′
N = 4 Homopolymer in Base Sequence Background
(near artificial wildtype) (100-mer)

(SEQ ID NO: 10)

5′-ACGTTGCTTT TTGACCTAGG GGAGCGTTGC GAAAATGGAT
ATGCCCCACC CATGGATAAA ATCGACGCCC CTTACGCCTA
AATGGGGCAG CGTTTTCTAA-3′
N = 5 Homopolymer in Base Sequence Background
(near artificial wildtype) (100-mer)

(SEQ ID NO: 11)

5′-ACGTTGCTTT TTGACCTAGG GGGCCGTTGC GAAAAAGGAT
ATCCCCCACC CATGGATAAA AACGACGCCC CCTACGCCTA
AAGGGGGCAG CTTTTTCTAA-3′
N = 6 Homopolymer in Base Sequence Background
(near artificial wildtype) (100-mer)

(SEQ ID NO: 12)

5′-ACGTTGTTTT TTGACCTAGG GGGGCGTTGC AAAAAAGGAT
ATCCCCCCTT CATGGTAAAA AACGACGCCC CCCACGCCTA
AGGGGGGCAG CTTTTTTGAA-3′
N = 7 Homopolymer in Base Sequence Background
(near artificial wildtype) (100-mer)

(SEQ ID NO: 13)

5′-ACGTTGTTTT TTTACCTAGG GGGGGATTGC AAAAAAAGAT
ATCCCCCCCT CATGGAAAAA AACGACGCCC CCCCAGCCTA
GGGGGGGCAG CTTTTTTTAA-3′
N = 8 Homopolymer in Base Sequence Background
(near artificial wildtype) (100-mer)

(SEQ ID NO: 14)

5′-ACGTTGTTTT TTTTCCTAGG GGGGGGTTGC AAAAAAAAGT
ATCCCCCCCC GATGGAAAAA AAAGACGCCC CCCCCGCCTG
GGGGGGGCAG TTTTTTTTAA-3′
N = 9 Homopolymer in Base Sequence Background
(near artificial wildtype) (100-mer)

(SEQ ID NO: 15)

5′-ACGTATTTTT TTTTCCTAGG GGGGGGGTGC AAAAAAAAAT
ATCCCCCCCC CATGGAAAAA AAAATGCCCC CCCCCGCCGG
GGGGGGGCAT TTTTTTTTAA-3′
N = 10 Homopolymer in Base Sequence Background
(near artificial wildtype) (100-mer)

(SEQ ID NO: 16)

5′-ACGATTTTTT TTTTCCTGGG GGGGGGGTGA AAAAAAAAAT
ATCCCCCCCC CCTGAAAAAA AAAATGCCCC CCCCCCAAGG
GGGGGGGGAT TTTTTTTTTA-3′
N = 11 Homopolymer in Base Sequence Background
(near artificial wildtype) (100-mer)

(SEQ ID NO: 17)

5′-ACGTTTTTTT TTTTCCGGGG GGGGGGGTGA AAAAAAAAAA
GCCCCCCCCC CCTAAAAAAA AAAATCCCCC CCCCCCAGGG
GGGGGGGGAT TTTTTTTTTT-3′
N = 12 Homopolymer in Base Sequence Background
(near artificial wildtype) (100-mer)

(SEQ ID NO: 18)

5′-ACTTTTTTTT TTTTCGGGGG GGGGGGGTAA AAAAAAAAAA
CCCCCCCCCC CCAAAAAAAA AAAACCCCCC CCCCCCGGGG
GGGGGGGGTT TTTTTTTTTT-3′
N = 13 Homopolymer in Base Sequence Background
(near artificial wildtype) (106-mer)

(SEQ ID NO: 19)

5′-ACTTTTTTTT TTTTTGGGGG GGGGGGGGAA AAAAAAAAAA+A
CCCCCCCCCC CC+CAAAAAAAA AAAA+ACCCCCC
CCCCCC+CGGGG GGGGGGGG+GTT TTTTTTTTTT+T-3′
(106-mer)

### Exemplary % AT DNA Control Panel Sequence (Flanked by NGS Platform-Specific Adaptors & Barcodes; not Shown):

1) Random Synthetic Sequence (100-mer)

0% AT Content in Base Sequence Background
(near artificial wildtype) (100-mer)

(SEQ ID NO: 20)

CGCGGCCGGC CGGCCGGCCGGCGCCGGCGC GCCGGCCGCG
CGCCGCGGCG GCGGCGCCGC CCGGCGCGCG GGCCGCGGCC
CGGCCGGCGC GCCCGCGCGG-3′
10% AT Content in Base Sequence Background
(near artificial wildtype) (100-mer)

(SEQ ID NO: 21)

CGCGGCCGGA CGGCCGGCCT GCGCCGGCGA GCCGGCCGCT
CGCCGCGGCA GCGGCGCCGT CCGGCGCGCA GGCCGCGGCT
CGGCCGGCGA GCCCGCGCGT-3′
20% AT Content in Base Sequence Background
(near artificial wildtype) (100-mer)

(SEQ ID NO: 22)

5′-AGCGGCCGGA TGGCCGGCCT ACGCCGGCGA TCCGGCCGCT
AGCCGCGGCA TCGGCGCCGT ACGGCGCGCA TGCCGCGGCT
AGGCCGGCGA TCCCGCGCGT-3′
30% AT Content in Base Sequence Background
(near artificial wildtype) (100-mer)

(SEQ ID NO: 23)

5′-AGCGGCCGAA TGGCCGGCTT ACGCCGGCAA TCCGGCCGTT
AGCCGCGGAA TCGGCGCCTT ACGGCGCGAA TGCCGCGGTT
AGGCCGGCAA TCCCGCGCTT-3′
40% AT Content in Base Sequence Background
(near artificial wildtype) (100-mer)

(SEQ ID NO: 24)

5′-AACGGCCGAA TTGCCGGCTT AAGCCGGCAA TTCGGCCGTT
AACCGCGGAA TTGGCGCCTT AAGGCGCGAA TTCCGCGGTT
AAGCCGGCAA TTCCGCGCTT-3′
50% AT Content in Base Sequence Background
(near artificial wildtype) (100-mer)

(SEQ ID NO: 25)

5′-AACGGCCAAA TTGCCGGTTT AAGCCGGAAA TTCGGCCTTT
AACCGCGAAA TTGGCGCTTT AAGGCGCAAA TTCCGCGTTT
AAGCCGGAAA TTCCGCGTTT-3′
60% AT Content in Base Sequence Background
(near artificial wildtype) (100-mer)

(SEQ ID NO: 26)

5′-AAAGGCCAAA TTTCCGGTTT AAACCGGAAA TTTGGCCTTT
AAACGCGAAA TTTGCGCTTT AAAGCGCAAA TTTCGCGTTT
AAACCGGAAA TTTCGCGTTT-3′
70% AT Content in Base Sequence Background
(near artificial wildtype) (100-mer)

(SEQ ID NO: 27)

5′-AAAGGCAAAA TTTCCGTTTT AAACCGAAAA TTTGGCTTTT
AAACGCAAAA TTTGCGTTTT AAAGCGAAAA TTTCGCTTTT
AAACCGAAAA TTTCGCTTTT-3′
80% AT Content in Base Sequence Background
(near artificial wildtype) (100-mer)

(SEQ ID NO: 28)

5′-AAAAGCAAAA TTTTCGTTTT AAAACGAAAA TTTTGCTTTT
AAAAGCAAAA TTTTCGTTTT AAAACGAAAA TTTTGCTTTT
AAAACGAAAA TTTTGCTTTT-3′
90% AT Content in Base Sequence Background
(near artificial wildtype) (100-mer)

(SEQ ID NO: 29)

5′-AAAAGAAAAA TTTTCTTTTT AAAACAAAAA TTTTGTTTTT
AAAAGAAAAA TTTTCTTTTT AAAACAAAAA TTTTGTTTTT
AAAACAAAAA TTTTGTTTTT-3′
100% AT Content in Base Sequence Background
(near artificial wildtype) (100-mer)

(SEQ ID NO: 30)

5′-AAAAAAAAAA TTTTTTTTTT AAAAAAAAAA TTTTTTTTTT
AAAAAAAAAA TTTTTTTTTT AAAAAAAAAA TTTTTTTTTT
AAAAAAAAAA TTTTTTTTTT-3′

### Exemplary % GC DNA Mutation Control Panel Sequence (Flanked by NGS Platform-Specific Adaptors & Barcodes; not Shown):

1) Random Synthetic Sequence (100-mer)

0% GC Content in Base Sequence Background
(near artificial wildtype) (100-mer)

(SEQ ID NO: 31)

5′-AATTATAATT AATATATTAT TAAATATAAT TAATATATTA
TTATATAAAT ATTATATAAT TAAATATTAT ATTTATATAA
ATTATATATA TATTATAATA-3′
10% GC Content in Base Sequence Background
(near artificial wildtype) (100-mer)

(SEQ ID NO: 32)

5′-AATTATAATC AATATATTAG TAAATATAAC TAATATATTG
TTATATAAAC ATTATATAAG TAAATATTAC ATTTATATAG
ATTATATATC TATTATAATG-3′
20% GC Content in Base Sequence Background
(near artificial wildtype) (100-mer)

(SEQ ID NO: 33)

5′-CATTATAATC GATATATTAG CAAATATAAC GAATATATTG
CTATATAAAC GTTATATAAG CAAATATTAC GTTTATATAG
CTTATATATC GATTATAATG-3′
30% GC Content in Base Sequence Background
(near artificial wildtype) (100-mer)

(SEQ ID NO: 34)

5′-CATTATAACC GATATATTGG CAAATATACC GAATATATGG
CTATATAACC GTTATATAGG CAAATATTCC GTTTATATGG
CTTATATACC GATTATAAGG-3′
40% GC Content in Base Sequence Background
(near artificial wildtype) (100-mer)

(SEQ ID NO: 35)

5′-CCTTATAACC GGTATATTGG CCAATATACC GGATATATGG
CCATATAACC GGTATATAGG CCAATATTCC GGTTATATGG
CCTATATACC GGTTATAAGG-3′
50% GC Content in Base Sequence Background
(near artificial wildtype) (100-mer)

(SEQ ID NO: 36)

5′-CCTTATACCC GGTATATGGG CCAATATCCC GGATATAGGG
CCATATACCC GGTATATGGG CCAATATCCC GGTTATAGGG
CCTATATCCC GGTTATAGGG-3′
60% GC Content in Base Sequence Background
(near artificial wildtype) (100-mer)

(SEQ ID NO: 37)

5′-CCCTATACCC GGGATATGGG CCCATATCCC GGGTATAGGG
CCCTATACCC GGGATATGGG CCCATATCCC GGGTATAGGG
CCCATATCCC GGGTATAGGG-3′
70% GC Content in Base Sequence Background
(near artificial wildtype) (100-mer)

(SEQ ID NO: 38)

5′-CCCTATCCCC GGGATAGGGG CCCATACCCC GGGTATGGGG
CCCTATCCCC GGGATAGGGG CCCATACCCC GGGTATGGGG
CCCATACCCC GGGTATGGGG-3′
80% GC Content in Base Sequence Background
(near artificial wildtype) (100-mer)

(SEQ ID NO: 39)

5′-CCCCATCCCC GGGGTAGGGG CCCCTACCCC GGGGATGGGG
CCCCATCCCC GGGGTAGGGG CCCCTACCCC GGGGATGGGG
CCCCTACCCC GGGGATGGGG-3′
90% GC Content in Base Sequence Background
(near artificial wildtype) (100-mer)

(SEQ ID NO: 40)

5′-CCCCACCCCC GGGGTGGGGG CCCCTCCCCC GGGGAGGGGG
CCCCACCCCC GGGGTGGGGG CCCCTCCCCC GGGGAGGGGG
CCCCTCCCCC GGGGAGGGGG-3′
100% GC Content in Base Sequence Background
(near artificial wildtype) (100-mer)

(SEQ ID NO: 41)

5′-CCCCCCCCCC GGGGGGGGGG CCCCCCCCCC GGGGGGGGGG
CCCCCCCCCC GGGGGGGGGG CCCCCCCCCC GGGGGGGGGG
CCCCCCCCCC GGGGGGGGGG-3′

### Exemplary Short Tandem Repeat DNA Panel Sequence (Flanked by NGS Platform-Specific Adaptors & Barcodes; not Shown):

Dinucleotide Repeats in Base Sequence Background (Artificial Wildtype) (200-mers)

Mono-Dinucleotide Repeats (200-mers)

(SEQ ID NO: 42)

5′-AAGTTGCATA ATGACCTAGG ACAGCGTTGC AGATCTGGAT
TAGCTTAACC TTTGGATCAA TCCGACGCGG TGTACGCCTA
AATTGGCCAG CGTTAGCTAA CAGTTGCATA CTGACCTAGG
CCAGCGTTGC CGATCTGGAT GAGCTTAACC GTTGGATCAA
GCCGACGCGG GGTACGCCTA AATTGGCCAG CGTTAGCTAA-3′
Doublet-Dinucleotide Repeats (200-mers)

(SEQ ID NO: 43)

5′-AAAATGCATA ATATCCTAGG ACACCGTTGC AGAGCTGGAT
TATATTAACC TTTTGATCAA TCTCACGCGG TGTGCGCCTA
AATTGGCCAG CGTTAGCTAA CACATGCATA CTCTCCTAGG
CCCCCGTTGC CGCGCTGGAT GAGATTAACC GTGTGATCAA
GCGCACGCGG GGGGCGCCTA AATTGGCCAG CGTTAGCTAA-3′
Triplet-Dinucleotide Repeats (200-mers)

(SEQ ID NO: 44)

5′-AAAAAACATA ATATATTAGG ACACACTTGC AGAGAGGGAT
TATATAAACC TTTTTTTCAA TCTCTCGCGG TGTGTGCCTA
AATTGGCCAG CGTTAGCTAA CACACACATA CTCTCTTAGG
CCCCCCTTGC CGCGCGGGAT GAGAGAAACC GTGTGTTCAA
GCGCGCGCGG GGGGGGCCTA AATTGGCCAG CGTTAGCTAA-3′
Quadruplex-Dinucleotide Repeats (200-mers)

(SEQ ID NO: 45)

5′-AAAAAAAATA ATATATATGG ACACACACGC AGAGAGAGAT
TATATATACC TTTTTTTTAA TCTCTCTCGG TGTGTGTGTA
AATTGGCCAG CGTTAGCTAA CACACACATA CTCTCTCTGG
CCCCCCCCGC CGCGCGCGAT GAGAGAGACC GTGTGTGTAA
GCGCGCGCGG GGGGGGGGTA AATTGGCCAG CGTTAGCTAA-3′
Quintiplex-Dinucleotide Repeats (200-mers)

(SEQ ID NO: 46)

5′-AAAAAAAAAA ATATATATAT ACACACACAC AGAGAGAGAG
TATATATATA TTTTTTTTTT TCTCTCTCGG TGTGTGTGTG
AATTGGCCAG CGTTAGCTAA CACACACACA CTCTCTCTCT
CCCCCCCCCC CGCGCGCGCG GAGAGAGAGA GTGTGTGTGT
GCGCGCGCGC GGGGGGGGGG AATTGGCCAG CGTTAGCTAA-3′

### Trinucleotide Repeats in Base Sequence Background (Artificial Wildtype)

A-Series Triplet Repeats (200-mers)

(SEQ ID NO: 47)

5′-AAATTGCATA AATACCTAGG AACGCGTTGC AAGTCTGGAT
ACACTTAACC ACTGGATCAA ACGGACGCGG ACCACGCCTA
ATATGGCCAG ATTTAGCTAA ATGTTGCATA ATCACCTAGG
AGAGCGTTGC AGTTCTGGAT AGGCTTAACC AGCGGATCAA
GCCGACGCGG GGTACGCCTA AATTGGCCAG CGTTAGCTAA-3′
T-Series Triplet Repeats (200-mers)

(SEQ ID NO: 48)

5′-TAATTGCATA TATACCTAGG TACGCGTTGC TAGTCTGGAT
TCACTTAACC TCTGGATCAA TCGGACGCGG TCCACGCCTA
TTATGGCCAG TTTTAGCTAA TTGTTGCATA TTCACCTAGG
TGAGCGTTGC TGTTCTGGAT TGGCTTAACC TGCGGATCAA
GCCGACGCGG GGTACGCCTA AATTGGCCAG CGTTAGCTAA-3′
C-Series Triplet Repeats (200-mers)

(SEQ ID NO: 49)

5′-CAATTGCATA CATACCTAGG CACGCGTTGC CAGTCTGGAT
CCACTTAACC CCTGGATCAA CCGGACGCGG CCCACGCCTA
CTATGGCCAG CTTTAGCTAA CTGTTGCATA CTCACCTAGG
CGAGCGTTGC CGTTCTGGAT CGGCTTAACC CGCGGATCAA
GCCGACGCGG GGTACGCCTA AATTGGCCAG CGTTAGCTAA-3′
G-Series Triplet Repeats (200-mers)

(SEQ ID NO: 50)

5′-GAATTGCATA GATACCTAGG GACGCGTTGC GAGTCTGGAT
GCACTTAACC GCTGGATCAA GCGGACGCGG GCCACGCCTA
GTATGGCCAG GTTTAGCTAA GTGTTGCATA GTCACCTAGG
GGAGCGTTGC GGTTCTGGAT GGGCTTAACC GGCGGATCAA
GCCGACGCGG GGTACGCCTA AATTGGCCAG CGTTAGCTAA-3′
Doublet A-Series Triplet Repeats (200-mers)

(SEQ ID NO: 51)

5′-AAAAAACATA AATAATTAGG AACAACTTGC AAGAAGGGAT
ACAACAAACC ACTACTTCAA ACGACGGCGG ACCACCCCTA
ATAATACCAG ATTATTCTAA ATGATGCATA ATCATCTAGG
AGAAGATTGC AGTAGTGGAT AGGAGGAACC AGCAGCTCAA
GCCGACGCGG GGTACGCCTA AATTGGCCAG CGTTAGCTAA-3′
Doublet T-Series Triplet Repeats (200-mers)

(SEQ ID NO: 52)

5′-TAATAACATA TATTATTAGG TACTACTTGC TAGTAGGGAT
TCATCAAACC TCTTCTTCAA TCGTCGGCGG TCCTCCCCTA
TTATTACCAG TTTTTTCTAA TTGTTGCATA TTCTTCTAGG
TGATGATTGC TGTTGTGGAT TGGTGGAACC TGCTGCTCAA
GCCGACGCGG GGTACGCCTA AATTGGCCAG CGTTAGCTAA-3′
Doublet C-Series Triplet Repeats (200-mers)

(SEQ ID NO: 53)

5′-CAACAACATA CATCATTAGG CACCACTTGC CAGCAGGGAT
CCACCAAACC CCTCCTTCAA CCGCCGGCGG CCCCCCCCTA
CTACTACCAG CTTCTTCTAA CTGCTGCATA CTCCTCTAGG
CGACGATTGC CGTCGTGGAT CGGCGGAACC CGCCGCTCAA
GCCGACGCGG GGTACGCCTA AATTGGCCAG CGTTAGCTAA-3′
Doublet G-Series Triplet Repeats (200-mers)

(SEQ ID NO: 54)

5′-GAAGAACATA GATGATTAGG GACGACTTGC GAGGAGGGAT
GCAGCAAACC GCTGCTTCAA GCGGCGGCGG GCCGCCCCTA
GTAGTACCAG GTTGTTCTAA GTGGTGCATA GTCGTCTAGG
GGAGGATTGC GGTGGTGGAT GGGGGGAACC GGCGGCTCAA
GCCGACGCGG GGTACGCCTA AATTGGCCAG CGTTAGCTAA-3′
Triplet A-Series Triplet Repeats (200-mers)

(SEQ ID NO: 55)

5′-AAAAAAAAAA AATAATAATG AACAACAACC AAGAAGAAGT
ACAACAACAC ACTACTACTA ACGACGACGG ACCACCACCA
ATAATTATAG ATTATTATTA ATGATGATGA ATCATCATCG
AGAAGAAGAC AGTAGTAGTT AGGAGGAGGC AGCAGCAGCA
GCCGACGCGG GGTACGCCTA AATTGGCCAG CGTTAGCTAA-3′
Triplet T-Series Triplet Repeats (200-mers)

(SEQ ID NO: 56)

5′-TAATAATAAA TATTATTATG TACTACTACC TAGTAGTAGT
TCATCATCAC TCTTCTTCTA TCGTCGTCGG TCCTCCTCCA
TTATTATTAG TTTTTTTTTA TTGTTGTTGA TTCTTCTTCG
TGATGATGAC TGTTGTTGTT TGGTGGTGGC TGCTGCTGCA
GCCGACGCGG GGTACGCCTA AATTGGCCAG CGTTAGCTAA-3′
Triplet C-Series Triplet Repeats (200-mers)

(SEQ ID NO: 57)

5′-CAACAACAAA CATCATCATG CACCACCACC CAGCAGCAGT
CCACCACCAC CCTCCTCCAA CCGCCGCCGG CCCCCCCCCA
CTACTACTAG CTTCTTCTTA CTGCTGCTGA CTCCTCCTCG
CGACGACGAC CGTCGTCGTT CGGCGGCGGC CGCCGCCGCA
GCCGACGCGG GGTACGCCTA AATTGGCCAG CGTTAGCTAA-3′
Triplet G-Series Triplet Repeats (200-mers)

(SEQ ID NO: 58)

5′-GAAGAAGAAA GATGATGATG GACGACGACC GAGGAGGAGT
GCAGCAGCAC GCTGCTGCTA GCGGCGGCGG GCCGCCGCCA
GTAGTAGTAG GTTGTTGTTA GTGGTGGTGA GTCGTCGTCG
GGAGGAGGAC GGTGGTGGTT GGGGGGGGGC GGCGGCGGCA
GCCGACGCGG GGTACGCCTA AATTGGCCAG CGTTAGCTAA-3′

### Exemplary Telomere Repeat Control Panel Sequence (Flanked by NGS Platform-Specific Adaptors & Barcodes; not Shown).

The sequences below were constructed for human, but the approach is also applicable to other telomere repeat sequences in other species (see Telomerase DB website; telomerase.asu.edu slash sequencestelomere.html; and table below).

Some known telomere nucleotide sequences

| | | Telomeric repeat |
|---|---|---|
| Group | Organism | (5′ to 3′ toward the end) |
| Vertebrates | Human, mouse, Xenopus | TTAGGG (SEQ ID NO: 95) |
| Filamentous fungi | Neurospora crassa | TTAGGG (SEQ ID NO: 96) |
| Slime moulds | Physarum, Didymium | TTAGGG (SEQ ID NO: 97) |
| | Dictyostelium | AG(1-8) (SEQ ID NO: 98) |
| Kinetoplastid protozoa | Trypanosoma, Crithidia | TTAGGG (SEQ ID NO: 99) |
| Ciliate protozoa | Tetrahymena, Glaucoma | TTGGGG (SEQ ID NO: 100) |
| | Paramecium | TTGGG(T/G) (SEQ ID NO: 101) |
| | Oxytricha, Stylonychia, | TTTTGGGG (SEQ ID NO: 102) |
| | Euplotes | |
| Apicomplexan | Plasmodium | TTAGGG(T/C) (SEQ ID NO: 103) |
| protozoa | | |
| Higher plants | Arabidopsis thaliana | TTTAGGG (SEQ ID NO: 104) |
| Green algae | Chlamydomonas | TTTTAGGG (SEQ ID NO: 105) |
| Insects | Bombyx mori | TTAGG (SEQ ID NO: 106) |
| Roundworms | Ascaris lumbricoides | TTAGGC (SEQ ID NO: 107) |
| Fission yeasts | Schizosaccharomyces pombe | TTAC(A)(C)G(1-8) (SEQ ID NO: 108) |
| Budding yeasts | Saccharomyces cerevisiae | TGTGGGTGTGGTG (from RNA template) (SEQ ID |
| | | NO: 109) |
| | | or G(2-3)(TG)(1-6)T (consensus) |
| | | (SEQ ID NO: 110) |
| | Saccharomyces castellii | TCTGGGTG (SEQ ID NO: 111) |
| | Candida glabrata | GGGGTCTGGGTGCTG (SEQ ID NO: 112) |
| | Candida albicans | GGTGTACGGATGTCTAACTTCTT (SEQ ID NO: 113) |
| | Candida tropicalis | GGTGTA[C/A]GGATGTCACGATCATT (SEQ ID |
| | | NO: 114) |
| | Candida maltosa | GGTGTACGGATGCAGACTCGCTT (SEQ ID NO: 115) |
| | Candida guillermondii | GGTGTAC (SEQ ID NO: 116) |
| | Candida pseudotropicalis | GGTGTACGGATTTGATTAGTTATGT (SEQ ID NO: 117) |
| | Kluyveromyces lactis | GGTGTACGGATTTGATTAGGTATGT (SEQ ID |
| | | NO: 118) |

In addition, the repeats can be designed from the 5′-end, expanding to the 3′-end (as opposed to the panel depicted; 3′-end, expanding to 5′-end).

1) Random Synthetic Sequence (100-mer)

N = 1 Sense Strand Telomere Repeat Base Sequence
(artificial wildtype) (100-mer)
(SEQ ID NO: 59)
5′-ACGTTGCATA CTGACCTAGG TAAGCGTTGC GAATCTGGAT ATGCTTAACC
CATGGATCAA TTCGACGCGG GTTACGCCTA AATTGGCCAG CGCGTTAGGG-3′
N = 2 Sense Strand Telomere Repeat Base Sequence
(artificial wildtype) (100-mer)
(SEQ ID NO: 60)
5′-ACGTTGCATA CTGACCTAGG TAAGCGTTGC GAATCTGGAT ATGCTTAACC
CATGGATCAA TTCGACGCGG GTTACGCCTA AATTGGCCTT AGGTTAGGG-3′
N = 3 Sense Strand Telomere Repeat Base Sequence
(artificial wildtype) (100-mer)
(SEQ ID NO: 61)
5′-ACGTTGCATA CTGACCTAGG TAAGCGTTGC GAATCTGGAT ATGCTTAACC
CATGGATCAA TTCGACGCGG GTTACGCCTA AATTAGGGTT AGGTTAGGG-3′
N = 4 Sense Strand Telomere Repeat Base Sequence
(artificial wildtype) (100-mer)
(SEQ ID NO: 62)
5′-ACGTTGCATA CTGACCTAGG TAAGCGTTGC GAATCTGGAT ATGCTTAACC
CATGGATCAA TTCGACGCGG GTTACGTTAG GGTTAGGGTT AGGTTAGGG-3′
N = 5 Sense Strand Telomere Repeat Base Sequence
(artificial wildtype) (100-mer)
(SEQ ID NO: 63)
5′-ACGTTGCATA CTGACCTAGG TAAGCGTTGC GAATCTGGAT ATGCTTAACC
CATGGATCAA TTCGACGCGG TTAGGGTTAG GGTTAGGGTT AGGTTAGGG-3′
N = 6 Sense Strand Telomere Repeat Base Sequence
(artificial wildtype) (100-mer)
(SEQ ID NO: 64)
5′-ACGTTGCATA CTGACCTAGG TAAGCGTTGC GAATCTGGAT ATGCTTAACC
CATGGATCAA TTCGTTAGGG TTAGGGTTAG GGTTAGGGTT AGGTTAGGG-3′
N = 7 Sense Strand Telomere Repeat Base Sequence
(artificial wildtype) (100-mer)
(SEQ ID NO: 65)
5′-ACGTTGCATA CTGACCTAGG TAAGCGTTGC GAATCTGGAT ATGCTTAACC
CATGGATCTT AGGGTTAGGG TTAGGGTTAG GGTTAGGGTT AGGTTAGGG-3′
N = 8 Sense Strand Telomere Repeat Base Sequence
(artificial wildtype) (100-mer)
(SEQ ID NO: 66)
5′-ACGTTGCATA CTGACCTAGG TAAGCGTTGC GAATCTGGAT ATGCTTAACC
CATTAGGGTT AGGGTTAGGG TTAGGGTTAG GGTTAGGGTT AGGTTAGGG-3′
N = 9 Sense Strand Telomere Repeat Base Sequence
(artificial wildtype) (100-mer)
(SEQ ID NO: 67)
5′-ACGTTGCATA CTGACCTAGG TAAGCGTTGC GAATCTGGAT ATGCTTTTAG
GGTTAGGGTT AGGGTTAGGG TTAGGGTTAG GGTTAGGGTT AGGTTAGGG-3′
N = 10 Sense Strand Telomere Repeat Base Sequence
(artificial wildtype) (100-mer)
(SEQ ID NO: 68)
5′-ACGTTGCATA CTGACCTAGG TAAGCGTTGC GAATCTGGAT TTAGGGTTAG
GGTTAGGGTT AGGGTTAGGG TTAGGGTTAG GGTTAGGGTT AGGTTAGGG-3′
N = 11 Sense Strand Telomere Repeat Base Sequence
(artificial wildtype) (100-mer)
(SEQ ID NO: 69)
5′-ACGTTGCATA CTGACCTAGG TAAGCGTTGC GAATTTAGGG TTAGGGTTAG
GGTTAGGGTT AGGGTTAGGG TTAGGGTTAG GGTTAGGGTT AGGTTAGGG-3′
N = 12 Sense Strand Telomere Repeat Base Sequence
(artificial wildtype) (100-mer)
(SEQ ID NO: 70)
5′-ACGTTGCATA CTGACCTAGG TAAGCGTTTT AGGGTTAGGG TTAGGGTTAG
GGTTAGGGTT AGGGTTAGGG TTAGGGTTAG GGTTAGGGTT AGGTTAGGG-3′
N = 13 Sense Strand Telomere Repeat Base Sequence
(artificial wildtype) (100-mer)
(SEQ ID NO: 71)
5′-ACGTTGCATA CTGACCTAGG TATTAGGGTT AGGGTTAGGG TTAGGGTTAG
GGTTAGGGTT AGGGTTAGGG TTAGGGTTAG GGTTAGGGTT AGGTTAGGG-3′
N = 14 Sense Strand Telomere Repeat Base Sequence
(artificial wildtype) (100-mer)
(SEQ ID NO: 72)
5′-ACGTTGCATA CTGACCTTAG GGTTAGGGTT AGGGTTAGGG TTAGGGTTAG
GGTTAGGGTT AGGGTTAGGG TTAGGGTTAG GGTTAGGGTT AGGTTAGGG-3′
N = 15 Sense Strand Telomere Repeat Base Sequence
(artificial wildtype) (100-mer)
(SEQ ID NO: 73)
5′-ACGTTGCATA TTAGGGTTAG GGTTAGGGTT AGGGTTAGGG TTAGGGTTAG
GGTTAGGGTT AGGGTTAGGG TTAGGGTTAG GGTTAGGGTT AGGTTAGGG-3′
N = 16 Sense Strand Telomere Repeat Base Sequence
(artificial wildtype) (100-mer)
(SEQ ID NO: 74)
5′-ACGTTTAGGG TTAGGGTTAG GGTTAGGGTT AGGGTTAGGG TTAGGGTTAG
GGTTAGGGTT AGGGTTAGGG TTAGGGTTAG GGTTAGGGTT AGGTTAGGG-3′
N = 17 Sense Strand Telomere Repeat Base Sequence
(artificial wildtype) (102-mer)
(SEQ ID NO: 75)
5′-TTAGGG TTAGGG TTAGGGTTAG GGTTAGGGTT AGGGTTAGGG
TTAGGGTTAG GGTTAGGGTT AGGGTTAGGG TTAGGGTTAG GGTTAGGGTT
AGGTTAGGG-3′
N = 18 Sense Strand Telomere Repeat Base Sequence
(artificial wildtype) (108-mer)
(SEQ ID NO: 76)
5′-TT AGGGTTAGGG TTAGGG TTAGGGTTAG GGTTAGGGTT AGGGTTAGGG
TTAGGGTTAG GGTTAGGGTT AGGGTTAGGG TTAGGGTTAG GGTTAGGGTT
AGGTTAGGG-3′
N = 1 Anti-Sense Strand Telomere Repeat Base Sequence
(artificial wildtype) (100-mer)
(SEQ ID NO: 77)
5′-ACGTTGCATA CTGACCTAGG TAAGCGTTGC GAATCTGGAT ATGCTTAACC
CATGGATCAA TTCGACGCGG GTTACGCCTA AATTGGCCAG CGCGCCCTAA-3′
N = 2 Anti-Sense Telomere Repeat Base Sequence
(artificial wildtype) (100-mer)
(SEQ ID NO: 78)
5′-ACGTTGCATA CTGACCTAGG TAAGCGTTGC GAATCTGGAT ATGCTTAACC
CATGGATCAA TTCGACGCGG GTTACGCCTA AATTGGCCCC CTAACCCTAA-3′
N = 3 Anti-Sense Telomere Repeat Base Sequence
(artificial wildtype) (100-mer)
(SEQ ID NO: 79)
5′-ACGTTGCATA CTGACCTAGG TAAGCGTTGC GAATCTGGAT ATGCTTAACC
CATGGATCAA TTCGACGCGG GTTACGCCTA AACCCTAACC CTAACCCTAA-3′
N = 4 Anti-Sense Telomere Repeat Base Sequence
(artificial wildtype) (100-mer)
(SEQ ID NO: 80)
5′-ACGTTGCATA CTGACCTAGG TAAGCGTTGC GAATCTGGAT ATGCTTAACC
CATGGATCAA TTCGACGCGG GTTACGCCCT AACCCTAACC CTAACCCTAA-3′
N = 5 Anti-Sense Telomere Repeat Base Sequence
(artificial wildtype) (100-mer)
(SEQ ID NO: 81)
5′-ACGTTGCATA CTGACCTAGG TAAGCGTTGC GAATCTGGAT ATGCTTAACC
CATGGATCAA TTCGACGCGG CCCTAACCCT AACCCTAACC CTAACCCTAA-3′
N = 6 Anti-Sense Telomere Repeat Base Sequence
(artificial wildtype) (100-mer)
(SEQ ID NO: 82)
5′-ACGTTGCATA CTGACCTAGG TAAGCGTTGC GAATCTGGAT ATGCTTAACC
CATGGATCAA TTCGCCCTAA CCCTAACCCT AACCCTAACC CTAACCCTAA-3′
N = 7 Anti-Sense Strand Telomere Repeat Base Sequence
(artificial wildtype) (100-mer)
(SEQ ID NO: 83)
5′-ACGTTGCATA CTGACCTAGG TAAGCGTTGC GAATCTGGAT ATGCTTAACC
CATGGATCCC CTAACCCTAA CCCTAACCCT AACCCTAACC CTAACCCTAA-3′
N = 8 Anti-Sense Telomere Repeat Base Sequence
(artificial wildtype) (100-mer)
(SEQ ID NO: 84)
5′-ACGTTGCATA CTGACCTAGG TAAGCGTTGC GAATCTGGAT ATGCTTAACC
CACCCTAACC CTAACCCTAA CCCTAACCCT AACCCTAACC CTAACCCTAA-3′
N = 9 Anti-Sense Telomere Repeat Base Sequence
(artificial wildtype) (100-mer)
(SEQ ID NO: 85)
5′-ACGTTGCATA CTGACCTAGG TAAGCGTTGC GAATCTGGAT ATGCTTCCCT
AACCCTAACC CTAACCCTAA CCCTAACCCT AACCCTAACC CTAACCCTAA-3′
N = 10 Anti-Sense Telomere Repeat Base Sequence
(artificial wildtype) (100-mer)
(SEQ ID NO: 86)
5′-ACGTTGCATA CTGACCTAGG TAAGCGTTGC GAATCTGGAT ATGCTTCCCT
AACCCTAACC CTAACCCTAA CCCTAACCCT AACCCTAACC CTAACCCTAA-3′
N = 11 Anti-Sense Telomere Repeat Base Sequence
(artificial wildtype) (100-mer)
(SEQ ID NO: 87)
5′-ACGTTGCATA CTGACCTAGG TAAGCGTTCC CTAATTAGGG TTAGGGTTAG
AACCCTAACC CTAACCCTAA CCCTAACCCT AACCCTAACC CTAACCCTAA-3′
N = 12 Anti-Sense Telomere Repeat Base Sequence
(artificial wildtype) (100-mer)
(SEQ ID NO: 88)
5′-ACGTTGCATA CTGACCTAGG TACCCTAACC CTAATTAGGG TTAGGGTTAG
AACCCTAACC CTAACCCTAA CCCTAACCCT AACCCTAACC CTAACCCTAA-3′
N = 13 Anti-Sense Telomere Repeat Base Sequence
(artificial wildtype) (100-mer)
(SEQ ID NO: 89)
5′-ACGTTGCATA CTGACCCCCT AACCCTAACC CTAATTAGGG TTAGGGTTAG
AACCCTAACC CTAACCCTAA CCCTAACCCT AACCCTAACC CTAACCCTAA-3′
N = 14 Anti-Sense Telomere Repeat Base Sequence
(artificial wildtype) (100-mer)
(SEQ ID NO: 90)
5′-ACGTTGCATA CTGACCCCCT AACCCTAACC CTAATTAGGG TTAGGGTTAG
AACCCTAACC CTAACCCTAA CCCTAACCCT AACCCTAACC CTAACCCTAA-3′
N = 15 Anti-Sense Telomere Repeat Base Sequence
(artificial wildtype) (100-mer)
(SEQ ID NO: 91)
5′-ACGTTGCATA CCCTAACCCT AACCCTAACC CTAATTAGGG TTAGGGTTAG
AACCCTAACC CTAACCCTAA CCCTAACCCT AACCCTAACC CTAACCCTAA-3′
N = 16 Anti-Sense Telomere Repeat Base Sequence
(artificial wildtype) (100-mer)
(SEQ ID NO: 92)
5′-ACGTCCCTAA CCCTAACCCT AACCCTAACC CTAATTAGGG TTAGGGTTAG
AACCCTAACC CTAACCCTAA CCCTAACCCT AACCCTAACC CTAACCCTAA-3′
N = 17 Anti-Sense Telomere Repeat Base Sequence
(artificial wildtype) (102-mer)
(SEQ ID NO: 93)
5′-CCCTAACCCTAA CCCTAACCCT AACCCTAACC CTAATTAGGG TTAGGGTTAG
AACCCTAACC CTAACCCTAA CCCTAACCCT AACCCTAACC CTAACCCTAA-3′
N = 18 Anti-Sense Telomere Repeat Base Sequence
(artificial wildtype) (108-mer)
(SEQ ID NO: 94)
5′-CCCTAACC CTAACCCTAA CCCTAACCCT AACCCTAACC CTAATTAGGG
TTAGGGTTAGAACCCTAACC CTAACCCTAA CCCTAACCCT AACCCTAACC
CTAACCCTAA-3′

Exemplary Centromere Repeat Control Panel Sequence (Flanked by NGS Platform-Specific Adaptors & Barcodes; not Shown).

The sequences below were constructed for human, but the approach is also applicable to other centromeric repeat sequences in other species In addition, the repeats can be designed from the 5′-end, expanding to the 3′-end (as opposed to the panel depicted; 3′-end, expanding to 5′-end).

1) Random Synthetic Sequence (100-mer)

N = 1 Sense Strand Centromere Repeat Base Sequence
(artificial wildtype) (100-mer)
(SEQ ID NO: 119)
5′-ACGTTGCATA CTGACCTAGG TAAGCGTTGC GAATCTGGAT ATGCTTAACC
CATGGATCAA TTCGACGCGG GTTACGCCTA AATTGGCCAG CGCGCTGGAA-3′
N = 2 Sense Strand Centromere Repeat Base Sequence
(artificial wildtype) (100-mer)
(SEQ ID NO: 120)
5′-ACGTTGCATA CTGACCTAGG TAAGCGTTGC GAATCTGGAT ATGCTTAACC
CATGGATCAA TTCGACGCGG GTTACGCCTA AATTGGCCAG TGGAATGGAA-3′
N = 3 Sense Strand Centromere Repeat Base Sequence
(artificial wildtype) (100-mer)
(SEQ ID NO: 121)
5′-ACGTTGCATA CTGACCTAGG TAAGCGTTGC GAATCTGGAT ATGCTTAACC
CATGGATCAA TTCGACGCGG GTTACGCCTA AATTGTGGAA TGGAATGGAA-3′
N = 4 Sense Strand Centromere Repeat Base Sequence
(artificial wildtype) (100-mer)
(SEQ ID NO: 122)
5′-ACGTTGCATA CTGACCTAGG TAAGCGTTGC GAATCTGGAT ATGCTTAACC
CATGGATCAA TTCGACGCGG GTTACGCCTA TGGAATGGAA TGGAATGGAA-3′
N = 5 Sense Strand Centromere Repeat Base Sequence
(artificial wildtype) (100-mer)
(SEQ ID NO: 123)
5′-ACGTTGCATA CTGACCTAGG TAAGCGTTGC GAATCTGGAT ATGCTTAACC
CATGGATCAA TTCGACGCGG GTTACTGGAA TGGAATGGAA TGGAATGGAA-3′
N = 6 Sense Strand Centromere Repeat Base Sequence
(artificial wildtype) (100-mer)
(SEQ ID NO: 124)
5′-ACGTTGCATA CTGACCTAGG TAAGCGTTGC GAATCTGGAT ATGCTTAACC
CATGGATCAA TTCGACGCGG TGGAA TGGAA TGGAATGGAA TGGAATGGAA-3′
N = 7 Sense Strand Centromere Repeat Base Sequence
(artificial wildtype) (100-mer)
(SEQ ID NO: 125)
5′-ACGTTGCATA CTGACCTAGG TAAGCGTTGC GAATCTGGAT ATGCTTAACC
CATGGATCAA TTCGATGGAA TGGAATGGAA TGGAATGGAA TGGAATGGAA-3′
N = 8 Sense Strand Centromere Repeat Base Sequence
(artificial wildtype) (100-mer)
(SEQ ID NO: 126)
5′-ACGTTGCATA CTGACCTAGG TAAGCGTTGC GAATCTGGAT ATGCTTAACC
CATGGATCAA TGGAATGGAA TGGAATGGAA TGGAATGGAA TGGAATGGAA-3′
N = 9 Sense Strand Centromere Repeat Base Sequence
(artificial wildtype) (100-mer)
(SEQ ID NO: 127)
5′-ACGTTGCATA CTGACCTAGG TAAGCGTTGC GAATCTGGAT ATGCTTAACC
CATGGTGGAA TGGAATGGAA TGGAATGGAA TGGAATGGAA TGGAATGGAA-3′
N = 10 Sense Strand Centromere Repeat Base Sequence
(artificial wildtype) (100-mer)
(SEQ ID NO: 128)
5′-ACGTTGCATA CTGACCTAGG TAAGCGTTGC GAATCTGGAT ATGCTTAACC
TGGAATGGAA TGGAATGGAA TGGAATGGAA TGGAATGGAA TGGAATGGAA-3′
N = 11 Sense Strand Centromere Repeat Base Sequence
(artificial wildtype) (100-mer)
(SEQ ID NO: 129)
5′-ACGTTGCATA CTGACCTAGG TAAGCGTTGC GAATCTGGAT ATGCTTGGAA
TGGAATGGAA TGGAATGGAA TGGAATGGAA TGGAATGGAA TGGAATGGAA-3′
N = 12 Sense Strand Centromere Repeat Base Sequence
(artificial wildtype) (100-mer)
(SEQ ID NO: 130)
5′-ACGTTGCATA CTGACCTAGG TAAGCGTTGC GAATCTGGAT TGGAATGGAA
TGGAATGGAA TGGAATGGAA TGGAATGGAA TGGAATGGAA TGGAATGGAA-3′
N = 13 Sense Strand Centromere Repeat Base Sequence
(artificial wildtype) (100-mer)
(SEQ ID NO: 131)
5′-ACGTTGCATA CTGACCTAGG TAAGCGTTGC GAATCTGGAA TGGAATGGAA
TGGAATGGAA TGGAATGGAA TGGAATGGAA TGGAATGGAA TGGAATGGAA-3′
N = 14 Sense Strand Centromere Repeat Base Sequence
(artificial wildtype) (100-mer)
(SEQ ID NO: 132)
5′-ACGTTGCATA CTGACCTAGG TAAGCGTTGC TGGAATGGAA TGGAATGGAA
TGGAATGGAA TGGAATGGAA TGGAATGGAA TGGAATGGAA TGGAATGGAA-3′
N = 15 Sense Strand Centromere Repeat Base Sequence
(artificial wildtype) (100-mer)
(SEQ ID NO: 133)
5′-ACGTTGCATA CTGACCTAGG TAAGCTGGAA TGGAATGGAA TGGAATGGAA
TGGAATGGAA TGGAATGGAA TGGAATGGAA TGGAATGGAA TGGAATGGAA-3′
N = 16 Sense Strand Centromere Repeat Base Sequence
(artificial wildtype) (100-mer)
(SEQ ID NO: 134)
5′-ACGTTGCATA CTGACCTAGG TGGAATGGAA TGGAATGGAA TGGAATGGAA
TGGAATGGAA TGGAATGGAA TGGAATGGAA TGGAATGGAA TGGAATGGAA-3′
N = 17 Sense Strand Centromere Repeat Base Sequence
(artificial wildtype) (100-mer)
(SEQ ID NO: 135)
5′-ACGTTGCATA CTGACTGGAA TGGAATGGAA TGGAATGGAA TGGAATGGAA
TGGAATGGAA TGGAATGGAA TGGAATGGAA TGGAATGGAA TGGAATGGAA-3′
N = 18 Sense Strand Centromere Repeat Base Sequence
(artificial wildtype) (100-mer)
(SEQ ID NO: 136)
5′-ACGTTGCATA TGGAATGGAA TGGAATGGAA TGGAATGGAA TGGAATGGAA
TGGAATGGAA TGGAATGGAA TGGAATGGAA TGGAATGGAA TGGAATGGAA-3′
N = 19 Sense Strand Centromere Repeat Base Sequence
(artificial wildtype) (100-mer)
(SEQ ID NO: 137)
5′-ACGTT TGGAA TGGAA TGGAA TGGAATGGAA TGGAATGGAA TGGAATGGAA
TGGAATGGAA TGGAATGGAA TGGAATGGAA TGGAATGGAA TGGAATGGAA-3′
N = 20 Sense Strand Centromere Repeat Base Sequence
(artificial wildtype) (100-mer)
(SEQ ID NO: 138)
5′-TGGAA TGGAA TGGAA TGGAA TGGAATGGAA TGGAATGGAA TGGAATGGAA
TGGAATGGAA TGGAATGGAA TGGAATGGAA TGGAATGGAA TGGAATGGAA-3′

### Anti-Sense Strand Centromere Repeat Base Sequence (Artificial Wildtype)

N = 1 Anti-Sense Centromere Repeat Base Sequence
(artificial wildtype) (100-mer)
(SEQ ID NO: 139)
5′-ACGTTGCATA CTGACCTAGG TAAGCGTTGC GAATCTGGAT ATGCTTAACC
CATGGATCAA TTCGACGCGG GTTACGCCTA AATTGGCCAG CGCGCTTCCA-3′
N = 2 Anti-Sense Centromere Repeat Base Sequence
(artificial wildtype) (100-mer)
(SEQ ID NO: 140)
5′-ACGTTGCATA CTGACCTAGG TAAGCGTTGC GAATCTGGAT ATGCTTAACC
CATGGATCAA TTCGACGCGG GTTACGCCTA AATTGGCCAG TTCCATTCCA-3′
N = 3 Anti-Sense Centromere Repeat Base Sequence
(artificial wildtype) (100-mer)
(SEQ ID NO: 141)
5′-ACGTTGCATA CTGACCTAGG TAAGCGTTGC GAATCTGGAT ATGCTTAACC
CATGGATCAA TTCGACGCGG GTTACGCCTA AATTGTTCCA TTCCATTCCA-3′
N = 4 Anti-Sense Centromere Repeat Base Sequence
(artificial wildtype) (100-mer)
(SEQ ID NO: 142)
5′-ACGTTGCATA CTGACCTAGG TAAGCGTTGC GAATCTGGAT ATGCTTAACC
CATGGATCAA TTCGACGCGG GTTACGCCTA TTCCATTCCA TTCCATTCCA-3′
N = 5 Anti-Sense Centromere Repeat Base Sequence
(artificial wildtype) (100-mer)
(SEQ ID NO: 143)
5′-ACGTTGCATA CTGACCTAGG TAAGCGTTGC GAATCTGGAT ATGCTTAACC
CATGGATCAA TTCGACGCGG GTTACTTCCA TTCCATTCCA TTCCATTCCA-3′
N = 6 Anti-Sense Centromere Repeat Base Sequence
(artificial wildtype) (100-mer)
(SEQ ID NO: 144)
5′-ACGTTGCATA CTGACCTAGG TAAGCGTTGC GAATCTGGAT ATGCTTAACC
CATGGATCAA TTCGACGCGG TTCCATTCCA TTCCATTCCA TTCCATTCCA-3′
N = 7 Anti-Sense Centromere Repeat Base Sequence
(artificial wildtype) (100-mer)
(SEQ ID NO: 145)
5′-ACGTTGCATA CTGACCTAGG TAAGCGTTGC GAATCTGGAT ATGCTTAACC
CATGGATCAA TTCGATTCCA TTCCATTCCA TTCCATTCCA TTCCATTCCA-3′
N = 8 Anti-Sense Centromere Repeat Base Sequence
(artificial wildtype) (100-mer)
(SEQ ID NO: 146)
5′-ACGTTGCATA CTGACCTAGG TAAGCGTTGC GAATCTGGAT ATGCTTAACC
CATGGATCAA TTCCATTCCA TTCCATTCCA TTCCATTCCA TTCCATTCCA-3′
N = 9 Anti-Sense Centromere Repeat Base Sequence
(artificial wildtype) (100-mer)
(SEQ ID NO: 147)
5′-ACGTTGCATA CTGACCTAGG TAAGCGTTGC GAATCTGGAT ATGCTTAACC
CATGGTTCCA TTCCATTCCA TTCCATTCCA TTCCATTCCA TTCCATTCCA-3′
N = 10 Anti-Sense Centromere Repeat Base Sequence
(artificial wildtype) (100-mer)
(SEQ ID NO: 148)
5′-ACGTTGCATA CTGACCTAGG TAAGCGTTGC GAATCTGGAT ATGCTTAACC
TTCCATTCCA TTCCATTCCA TTCCATTCCA TTCCATTCCA TTCCATTCCA-3′
N = 11 Anti-Sense Centromere Repeat Base Sequence
(artificial wildtype) (100-mer)
(SEQ ID NO: 149)
5′-ACGTTGCATA CTGACCTAGG TAAGCGTTGC GAATCTGGAT ATGCTTTCCA
TTCCATTCCA TTCCATTCCA TTCCATTCCA TTCCATTCCA TTCCATTCCA-3′
N = 12 Anti-Sense Centromere Repeat Base Sequence
(artificial wildtype) (100-mer)
(SEQ ID NO: 150)
5′-ACGTTGCATA CTGACCTAGG TAAGCGTTGC GAATCTGGAT TTCCATTCCA
TTCCATTCCA TTCCATTCCA TTCCATTCCA TTCCATTCCA TTCCATTCCA-3′
N = 13 Anti-Sense Centromere Repeat Base Sequence
(artificial wildtype) (100-mer)
(SEQ ID NO: 151)
5′-ACGTTGCATA CTGACCTAGG TAAGCGTTGC GAATCTTCCA TTCCATTCCA
TTCCATTCCA TTCCATTCCA TTCCATTCCA TTCCATTCCA TTCCATTCCA-3′
N = 14 Anti-Sense Centromere Repeat Base Sequence
(artificial wildtype) (100-mer)
(SEQ ID NO: 152)
5′-ACGTTGCATA CTGACCTAGG TAAGCGTTGC TTCCATTCCA TTCCATTCCA
TTCCATTCCA TTCCATTCCA TTCCATTCCA TTCCATTCCA TTCCATTCCA-3′
N = 15 Anti-Sense Centromere Repeat Base Sequence
(artificial wildtype) (100-mer)
(SEQ ID NO: 153)
5′-ACGTTGCATA CTGACCTAGG TAAGCTTCCA TTCCATTCCA TTCCATTCCA
TTCCATTCCA TTCCATTCCA TTCCATTCCA TTCCATTCCA TTCCATTCCA-3′
N = 16 Anti-Sense Centromere Repeat Base Sequence
(artificial wildtype) (100-mer)
(SEQ ID NO: 154)
5′-ACGTTGCATA CTGACCTAGG TTCCATTCCA TTCCATTCCA TTCCATTCCA
TTCCATTCCA TTCCATTCCA TTCCATTCCA TTCCATTCCA TTCCATTCCA-3′
N = 17 Anti-Sense Centromere Repeat Base Sequence
(artificial wildtype) (100-mer)
(SEQ ID NO: 155)
5′-ACGTTGCATA CTGACcTTCCA TTCCATTCCA TTCCATTCCA TTCCATTCCA
TTCCATTCCA TTCCATTCCA TTCCATTCCA TTCCATTCCA TTCCATTCCA-3′
N = 18 Anti-Sense Centromere Repeat Base Sequence
(artificial wildtype) (100-mer)
(SEQ ID NO: 156)
5′-ACGTTGCATA TTCCATTCCA TTCCATTCCA TTCCATTCCA TTCCATTCCA
TTCCATTCCA TTCCATTCCA TTCCATTCCA TTCCATTCCA TTCCATTCCA-3′
N = 19 Anti-Sense Centromere Repeat Base Sequence
(artificial wildtype) (100-mer)
(SEQ ID NO: 157)
5′-ACGTTTTCCA TTCCATTCCA TTCCATTCCA TTCCATTCCA TTCCATTCCA
TTCCATTCCA TTCCATTCCA TTCCATTCCA TTCCATTCCA TTCCATTCCA-3′
N = 20 Anti-Sense Centromere Repeat Base Sequence
(artificial wildtype) (100-mer)
(SEQ ID NO: 158)
5′-TTCCATTCCA TTCCATTCCA TTCCATTCCA TTCCATTCCA TTCCATTCCA
TTCCATTCCA TTCCATTCCA TTCCATTCCA TTCCATTCCA TTCCATTCCA-3′

### Exemplary Copy Number Variation DNA Control Calibration Panel Sequences (Flanked by NGS Platform-Specific Adaptors & Barcodes; not Shown).

Copy Number Variation (CNV) panels find use as artificial internal control sequences to monitor the inherent sensitivity of NGS based digital molecular counting applications. Exemplary applications in oncology include detection of chromosome aneuploidy and copy number imbalance (CNVs) in cancer, and determining the copy number status of a focal gene amplification in cancer (e.g. Her-2 gene amplification in breast cancer). In these instances, gene and/or chromosome copy number varies over a modest range between zero and approximately 100 copies, and differs by single copy (whole copy) increments. Other applications require more sensitive limits of detection to enable accurate and precise measurement of fractional copies (less than a single copy). Non-invasive fetal aneuploidy detection directly from cell-free fetal DNA circulating in maternal blood is an example for ultra-sensitive detection of fractional copy number changes (˜0.02-0.05). For a case of fetal trisomy (e.g. trisomy 21), at 10% cell-free fetal DNA plasma concentrations, the fractional abundance of Chr-21 derived fetal DNA over maternal Chr-21 derived DNA is 1.05 (Lo et. al. 2007 PNAS 104 (32): 13116-13121). At the other spectrum, an example of a molecular counting application that requires a wide linear dynamic range is gene expression analysis, since natural RNA abundances in cells can vary from single individual transcripts to millions of RNA copies per cell.

In some embodiments, CNV panels comprise synthetic oligonucleotides with unique 5′-20-mer tag sequences mixed at pre-defined stoichiometric ratios and concentrations (calibration panel). The number of unique tag sequences used can be tailored for the desired application. For example, one may desire an RNA expression analysis control panel that covers a linear 6 log dynamic range, at specified log-fold increments (7 tags; mixed at 1, 10, 100, 1000, 10,000, 100,000, 1,000,000 copies), a DNA CNV panel that covers a couple of logs of linear dynamic range at single copy resolution (100 tags; mixed at 1 through 100 copies, inclusive in single copy increments), or an ultra-sensitive fetal DNA aneuploidy (fractional copy) panel that covers one-tenth of a log of linear dynamic range (10 tags; 1.01, 1.02, 1.03, 1.04, 1.05, 1.06, 1.07, 1.08, 1.09, 1.10 molar ratio). Flexibility exists to design the desired number of tag sequences across a specified, pre-determined number of concentrations; creating a custom titration series for tuning the desired dynamic range and calibrating the desired performance and sensitivity.

The panel below represents an embodiment of an exemplary CNV control panel composed of 4 separate uniquely tagged oligonucleotides (Seq A, Seq B, Seq C, and Seq D), at pre-defined stoichiometry (molar ratio), and designed to cover a 2-log range with added low-end sensitivity to enable ultra-sensitive fractional copy analysis.

Panel comprises 4 synthetic oligos (Seq A, Seq B, Seq C, and Seq D) with unique 5′-20-mer tag sequences mixed at pre-defined stoichiometric ratios and concentrations.

### 100 Copies Seq A+10 Copies Seq B+1 Copy Seq C+1.05 Copies Seq D

1) 100 Copy Random Synthetic Tag Sequence A (100-mer)

20-mer Tag Sequence (used for molecular CNV counting)+80-mer Distal Artificial Target Sequence

(SEQ ID NO: 159)

5′-TCTGATTCAG CTAGTCCAGCTAAGCGTTGC GAATCTGGAT
ATGCTTAACC CATGGATCAA TTCGACGCGG GTTACGCCTA
AATTGGCCAG CGTTAGCTAA-3′

2) 10 Copy Random Synthetic Tag Sequence B (100-mer)

20-mer Tag Sequence (used for Molecular CNV counting)+80-mer Distal Artificial Target Sequence

(SEQ ID NO: 160)

5′-CTGTCGGTAT AGCAGAATCGTAAGCGTTGC GAATCTGGAT
ATGCTTAACC CATGGATCAA TTCGACGCGG GTTACGCCTA
AATTGGCCAG CGTTAGCTAA-3′

3) Single Copy Random Synthetic Tag Sequence C (100-mer)

20-mer Tag Sequence (used for Molecular CNV counting)+80-mer Distal Artificial Target Sequence

(SEQ ID NO: 161)

5′-AGCATCAAGC TCTGCATGCCTAAGCGTTGC GAATCTGGAT
ATGCTTAACC CATGGATCAA TTCGACGCGG GTTACGCCTA
AATTGGCCAG CGTTAGCTAA-3′

4) Fractional Copy (1.05) Random Synthetic Tag Sequence D (100-mer)

20-mer Tag Sequence (used for molecular CNV counting)+80-mer Distal Artificial Target Sequence

(SEQ ID NO: 162)

5′-GATCGACACT GATCAGACAGTAAGCGTTGC GAATCTGGAT
ATGCTTAACC CATGGATCAA TTCGACGCGG GTTACGCCTA
AATTGGCCAG CGTTAGCTAA-3′

### Example 2

### Control Panels for Next-Generation Sequencing

During the development of embodiments of the technology provided herein, experiments were conducted to test embodiments of a nucleic acid control panel as described herein for monitoring next generation sequencing (NGS) run and/or system performance. In particular, panels of oligonucleotides were designed to measure the performance of next generation sequencing systems and/or runs. The panel was designed to allow for the assessment of a NGS system and/or run across a range of oligonucleotide sequence content (e.g., oligonucleotides comprising a range of nucleotide sequence features, sizes, structures, concentrations, etc.). A subset of the NGS control panel oligonucleotides was selected and run on a sequencer apparatus (Ion Torrent PGM sequencer).

The control panel oligonucleotide subset comprised different oligonucleotides or oligonucleotide subsets to allow for the assessment of NGS system performance across different performance criteria such as, e.g., identifying SNPs at varying dilutions of sample, sequencing homopolymers, detecting DNA copy number, and sequencing samples comprising various % GC contents. A total of 13 control panel oligonucleotides were synthesized (Integrated DNA Technologies) and sequenced on the sequencing apparatus. The sequences of the control panel oligonucleotides that were assessed in these experiments are listed below. The terms “SeqID” and “Oligo” are used throughout this example to refer to individual oligonucleotides of the various control panel oligonucleotides (the term SeqID is not to be confused with the SEQ ID NO: identifiers associated with sequences provided herein). All nucleotide sequences of oligonucleotides are written in a 5 prime to 3 prime direction.

### A—Somatic DNA Control Panel for SNPs

These oligos were tested at various dilutions (e.g., 1:10, 1:100, 1:1000, 1:10000) to test SNP detection by NGS

Oligo 1

(SEQ ID NO: 163)

ACGTTGCATA CTGACCTAGG TAAGCGTTGC GAATCTGGAT
ATGCTTAACC CATGGATCAA TTCGACGCGG GTTACGCCTA
AATTGGCCAG CGTTAGCTAA
Oligo 2

(SEQ ID NO: 164)

ACGTTGCATG CTGACCTAGG TAAGCGTTGC GAATCTGGAT
CTGCTTAACC CATGGATCAC TTCGACGCGG GTTACGCCTA
AATTGGCCTG CGTTAGCTAA
Oligo 3

(SEQ ID NO: 165)

ACGTTGCATC CTGACCTAGG TAAGCGTTGC GAATCTGGAT
TTGCTTAACC CATGGATCAT TTCGACGCGG GTTACGCCTA
AATTGGCCCG CGTTAGCTAA
Oligo 4

(SEQ ID NO: 166)

ACGTTGCATT CTGACCTAGG TAAGCGTTGC GAATCTGGAT
GTGCTTAACC CATGGATCAG TTCGACGCGG GTTACGCCTA
AATTGGCCGG CGTTAGCTAA

### B—Homopolymers

N = 4 repeats (AAAA, GGGG, CCCC, TTTT)
Oligo 10

(SEQ ID NO: 167)

ACGTTGCTTT TTGACCTAGG GGAGCGTTGC GAAAATGGAT
ATGCCCCACC CATGGATAAA ATCGACGCCC CTTACGCCTA
AATGGGGCAG CGTTTTCTAA

### C—DNA Copy Number Variation (CNV)

These oligos were tested at different molar ratios, e.g., at 5-fold and 1.5-fold ratios

Oligo 159

(SEQ ID NO: 168)

TCTGATTCAG CTAGTCCAGC TAAGCGTTGC GAATCTGGAT
ATGCTTAACC CATGGATCAA TTCGACGCGG GTTACGCCTA
AATTGGCCAG CGTTAGCTAA
Oligo 160

(SEQ ID NO: 169)

CTGTCGGTAT AGCAGAATCG TAAGCGTTGC GAATCTGGAT
ATGCTTAACC CATGGATCAA TTCGACGCGG GTTACGCCTA
AATTGGCCAG CGTTAGCTAA
Oligo 161

(SEQ ID NO: 170)

AGCATCAAGC TCTGCATGCC TAAGCGTTGC GAATCTGGAT
ATGCTTAACC CATGGATCAA TTCGACGCGG GTTACGCCTA
AATTGGCCAG CGTTAGCTAA
Oligo 162

(SEQ ID NO: 171)

GATCGACACT GATCAGACAG TAAGCGTTGC GAATCTGGAT
ATGCTTAACC CATGGATCAA TTCGACGCGG GTTACGCCTA
AATTGGCCAG CGTTAGCTAA

### D—% GC Content

These oligos were tested comprising various amounts of G and C nucleotides, e.g., at 60% & 70% GC content

Oligo 37

(SEQ ID NO: 172)

CCCTATACCC GGGATATGGG CCCATATCCC GGGTATAGGG
CCCTATACCC GGGATATGGG CCCATATCCC GGGTATAGGG
CCCATATCCC GGGTATAGGG
Oligo 38

(SEQ ID NO: 173)

CCCTATCCCC GGGATAGGGG CCCATACCCC GGGTATGGGG
CCCTATCCCC GGGATAGGGG CCCATACCCC GGGTATGGGG
CCCATACCCC GGGTATGGGG
Oligo 26

(SEQ ID NO: 174)

AAAGGCCAAA TTTCCGGTTT AAACCGGAAA TTTGGCCTTT
AAACGCGAAA TTTGCGCTTT AAAGCGCAAA TTTCGCGTTT
AAACCGGAAA TTTCGCGTTT
Oligo 27

(SEQ ID NO: 175)

AAAGGCAAAA TTTCCGTTTT AAACCGAAAA TTTGGCTTTT
AAACGCAAAA TTTGCGTTTT AAAGCGAAAA TTTCGCTTTT
AAACCGAAAA TTTCGCTTTT

Adapter sequences (Ion Torrent A and P1) were added to the above control panel (test) oligonucleotides for introduction into the workflow of sequencer apparatus (PGM OneTouch2 emPCR) instrument. The test oligonucleotides were 184 bp long after the addition of the adaptors; these oligonucleotides comprising a test sequence and adaptors are called “ultramers” herein. After adaptor addition, the composition of each ultramer was:
5′-(Ion Xpress Barcoded A Adapter)-[Oligo]-(P1 Adapter)-3′
   The sequences of the adaptors are:

Ion Xpress Barcoded A Adapter

(SEQ ID NO: 176)

CCATCTCATCCCTGCGTGTCTCCGACTCAGCTAAGGTAACGAT
P1 Adapter

(SEQ ID NO: 177)

ATCACCGACTGCCCATAGAGAGGAAAGCGGAGGCGTAGTGG

The Ion Xpress Barcoded A Adapter is the oligonucleotide named “IonXpress_{—}001” for all 13 oligonucleotides. The sequence for the IonXpress_{—}001 barcode is CTAAGGTAAC (SEQ ID NO: 178) and is underlined above.

The experiments described below were performed with the following reagents and materials unless noted otherwise: Ion Plus Fragment Library Kit (Ion Torrent catalog number 4471252, lot number 017C02-13); Ampure XP Reagent (Beckman Coulter catalog number A63880, lot number 14403400); Ion PGM 200 v2 Sequencing Kit (Ion Torrent catalog number 4482008, lot number 053B09-13); Ion OneTouch2 200 Reagents Kit (Ion Torrent catalog number 4481107, lot number 058B03-12); Dynabeads MyOne Streptavidin C1 (Invitrogen catalog number 650.01, lot number 94749830); Ion PGM v2 316 Chip (Ion Torrent catalog number 4483188, lot number 1114586); Bioanalyzer High Sensitivity DNA Reagents (Agilent catalog number 5067-4626, lot number 1310); Molecular Biology Grade Water (Invitrogen catalog number 10977-015, lot number 1292609); Buffer EB (Qiagen catalog number 1014609, lot number 433160715). Instruments used were the following unless noted otherwise: Ion Torrent PGM, Ion Torrent OneTouch2, Ion Torrent Enrichment Station, Bioanalyzer 2100, and an ABI 9700 Thermocycler (GeneAmp PCR System 9700).

During the development of embodiments of the technology described herein, experiments were conducted according to the following methods. Each 184-mer control panel ultramer was made double-stranded (to provide a “ds ultramer”) by performing 5 cycles of amplification using PCR reagents and manufacturer's instructions (e.g., a protocol from the Life Technologies Ion Plus Fragment Library Kit (Cat. no. 4471252)). Double-stranded ultramers were purified using a solid-support purification method (1:2 Ampure XP bead purification). Purification was performed two times. Double-stranded (ds) ultramer concentrations were measured using BioAnalyzer High-sensitivity chips. Ion Torrent OneTouch2 (emPCR) runs were performed following the “Ion PGM Template OT2 200 Kit User Guide”. The Ion Torrent OneTouch2 amplification mix was prepared by mixing double-stranded control panel ultramers with an Ion torrent-adapted Lung Panel library at a 1:1 molar ratio for a total concentration of 26 pM in 25 uL. The total OneTouch amplification mix library concentration was 650 fM (e.g., 25 uL/1000 uL×26 pM). The Lung Panel library was generated using a Lung Panel 20-plex primer mix (Abbott Molecular) with 10 ng of a Horizon Diagnostics Quantitative Multiplex Reference Standard (Cat#HD700) following the Short Amplicon Prep Ion Plus Fragment Library Kit user guide. The amount of each ultramer combined with the AM Lung Panel Horizon library is shown below in Table 1:

TABLE 1
test samples comprising ultramers

| | | Concentration | Volume | | |
|---|---|---|---|---|---|
| | | Used to | Used to | | |
| | Ion Xpress | create mix | create Mix | Concentration | Volume added |
| Library/ds Ultramer | Barcode | (pM) | (uL) | (pM) | (uL) |
| Oligo1 | IonXpress_001 | 100 | 2 | 27.775 pM | 1.8 from |
| Oligo2 | IonXpress_001 | 10 | 2 | oligo1-4 | Oligo1-4 |
| Oligo3 | IonXpress_001 | 1 | 2 | sum | mix |
| Oligo4 | IonXpress_001 | 0.1 | 2 | | |
| Oligo10 | IonXpress_001 | n/a | n/a | 26 | 1.8 |
| Oligo159 | IonXpress_001 | 50 | 2 | 26.250 pM | 1.8 from |
| Oligo160 | IonXpress_001 | 30 | 2 | oligo159-162 | Oligo159-162 |
| Oligo161 | IonXpress_001 | 15 | 2 | sum | mix |
| Oligo162 | IonXpress_001 | 10 | 2 | | |
| Oligo37 | IonXpress_001 | n/a | n/a | 26 | 1.8 |
| Oligo38 | IonXpress_001 | n/a | n/a | 26 | 1.8 |
| Oligo26 | IonXpress_001 | n/a | n/a | 26 | 1.8 |
| Oligo 27 | IonXpress_001 | n/a | n/a | 26 | 1.8 |
| AM 20plex Lung | IonXpress_013 | n/a | n/a | 26 | 12.5 |
| Panel Library | | | | | |
| (template = Horizon | | | | | |
| Quantitative Multiplex | | | | | |
| Reference Standard) | | | | | |
| | | | | Total: | 25 uL |

Sequencing runs were performed on the sequencing apparatus (Ion Torrent PGM) using Ion 316 chips following the Ion PGM™ Sequencing 200 Kit v2 User Guide. Two PGM 316 chip runs were performed.

Ion Torrent Suite FASTQ files corresponding to the control panel (IonXpress barcode 001) or 20-plex Lung Panel library (IonXpress barcode 013) were analyzed using bioinformatics software (CLC Genomics Workbench), e.g., using the ‘Map Reads to Reference’ function. Variants present in the 20-plex Lung Panel library were called using the CLC Genomics Workbench ‘Quality based variant detection’ function. For the control panel output, the reference for alignment was the 100-mer sequence of the appropriate oligonucleotide from the 13 control panel oligonucleotides. For the 20-plex Lung panel library, the reference for alignment was the sequence of the 20 panel amplicons. CLC Genomics Workbench aligner and variant caller parameters are shown below:

References=Ctrl_Panel_Reference

Masking mode=No Masking

Mismatch cost=2

Insertion cost=3

Deletion cost=3

Length fraction=0.5

Similarity fraction=0.8

Global alignment=Yes

Non-specific match handling=Map randomly

Output mode=Create stand-alone read mappings

Create report=Yes

Collect un-mapped reads=No

Neighborhood radius=5

Maximum gap and mismatch count=2

Minimum neighborhood quality=15

Minimum central quality=20

Ignore non-Specific matches=Yes

Ignore broken pairs=Yes

Minimum coverage=10

Minimum variant frequency (%)=0.5

Maximum expected alleles=2

Advanced=No

Require presents in both forward and reverse reads=yes

Ignore variants in non-specific regions=No

Filter 454/Ion homopolymer indels=No

Create track=Yes

Create annotated table=Yes

Genetic code=1 standard

Results

During the development of embodiments of the technology described herein, data were collected from testing the Somatic DNA control panel for SNP detection. Table 2 shows the dilutions of Oligos 1-4 that were used in the experiments.

TABLE 2
concentrations of Oligos 1-4 used

| | Concentration | | | |
|---|---|---|---|---|
| | in 1000 μl | | NGS | Number |
| | PGM OneTouch | expected | determined | of NGS |
| Name | emPCR amplifi- | % compared | % compared | mapped |
| (dilution) | cation mix (fM) | to Oligo 1 | to Oligo 1 | reads |
| Oligo 1 | 45 | — | — | 94,758 |
| Oligo 2 | 4.5 | 10.00% | 7.82% | 7411 |
| (1:10) | | | | |
| Oligo 3 | 0.45 | 1.00% | 0.71% | 669 |
| (1:100) | | | | |
| Oligo 4 | 0.045 | 0.10% | 0.25% | 238 |
| (1:1000) | | | | |

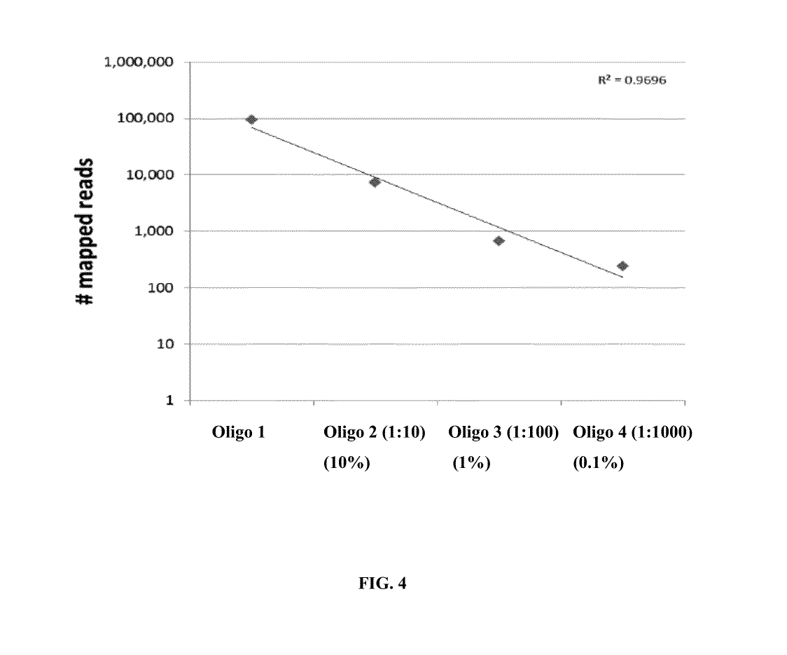

Data were plotted to show the NGS read counts across the titration of SNP-containing oligonucleotides (control panel Oligos 1-4). The data indicate a SNP detection sensitivity of 10% and 1% (FIG. 4).

Table 3 (below) shows the percent of several variants detected in the Lung Panel library that was generated using the multiplex reference standard (Horizon Quantitative Multiplex Standard; see Table 1). This Lung Panel library was from the same NGS run that contained the SNP containing control panel oligonucleotides shown in FIG. 4.

TABLE 3
% of variants detected in the quantitative multiplex
reference standard (Horizon Standard)

| | | | Horizon | |
|---|---|---|---|---|
| | | | Provided/ | AM 20plex |
| | | | Expected | PGM Run |
| | | | Allelic | Allelic |
| Chromosome | Gene | Variant | Frequency | Frequency |
| 7q34 | BRAF | V600E | 10.5% | 10.3% |
| 7p12 | EGFR | ΔE746-A750 | 2.0% | 1.2% |
| 7p12 | EGFR | L858R | 3.0% | 1.3% |
| 7p12 | EGFR | T790M | 1.0% | 0.9% |
| 7p12 | EGFR | G719S | 24.5% | 27.9% |
| 12p12.1 | KRAS | G13D | 15.0% | 16.0% |
| 12p12.1 | KRAS | G12D | 6.0% | 9.2% |
| 3q26.3 | PI3KCA | H1047R | 17.5% | 17.2% |
| 3q26.3 | PI3KCA | E545K | 9.0% | 8.5% |

Further, during the development of embodiments of the technology described herein, data were collected from testing the homopolymer test oligonucleotide (Oligo 10). Table 4 (below) shows the performance of Oligo 10. In some embodiments, it is contemplated that Oligo 10 is used in an NGS control panel to assess homopolymer sequencing performance between NGS systems or runs.

TABLE 4
Control panel Oligo 10/Homopolymer performance

| | # SeqID 10 Reads |
|---|---|
| # Perfect Reads | 13,310 |
| # Reads @ 99% accuracy | 17,625 |
| # Reads @ 98% accuracy | 50,041 |
| # total reads | 82,026 |
| | % SeqID 10 Reads |
| % Perfect Reads | 16.2% |
| % Reads @ 99% accuracy | 21.5% |
| % Reads @ 98% accuracy | 61.0% |
| % total reads | 100.0% |

Next, during the development of embodiments of the technology described herein, experiments were conducted to assess the performance of NGS to detect DNA copy number variation. In particular, Oligos 159, 160, 161, and 162 were tested at different molar ratios of 5-fold, 3-fold, 1.5-fold, and 1-fold. Table 5 shows the concentrations of test Oligos, copies expected to be detected, the number of mapped reads for each Oligo, and the measured number of copies relative to the Oligo provided at 1× concentration (Oligo 162).

TABLE 5
Oligo 159-162 dilutions performed and NGS mapped outputs

| | Concentration in 1000 uL | Expected | | NGS determined |
|---|---|---|---|---|
| | PGM OneTouch empPCR | Copies compared | # NGS mapped | copies compared |
| Name | Amplification Mix (fM) | to SeqID162 | reads | to SeqID162 |
| SeqID 159 | 22.50 | 5X | 57,446 | 6.1 |
| SeqID 160 | 13.50 | 3X | 31,404 | 3.4 |
| SeqID 161 | 6.75 | 1.5X | 12,856 | 1.4 |
| SeqID 162 | 4.50 | 1X | 9,361 | — |

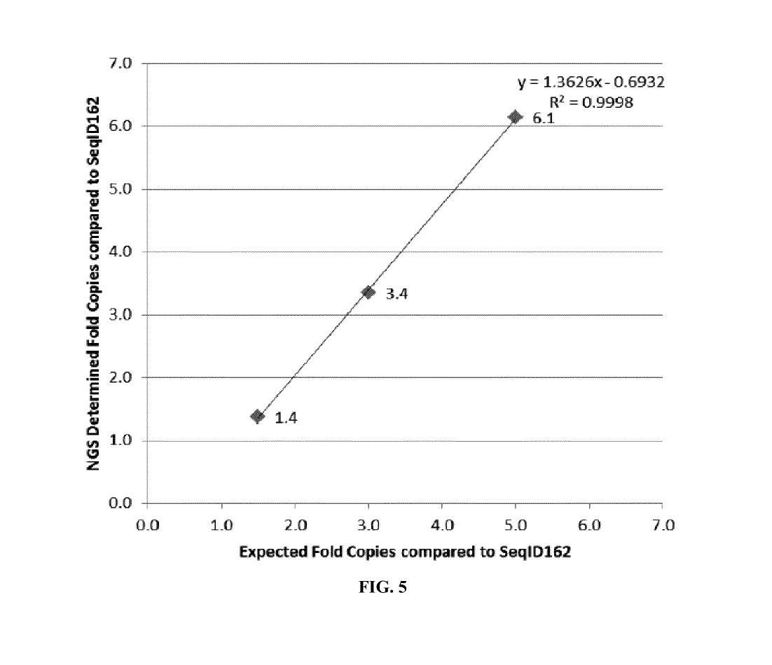

Data collected were plotted to show the determined copy number versus the expected copy number (FIG. 5).

During the development of the technology provided herein, experiments were conducted to test the performance of NGS to provide sequence from templates comprising % GC contents of various amounts. Table 6 shows the results of these experiments.

TABLE 6
Control Panel Oligo 37 (60% GC) & Oligo 38 (70% GC)

| | # SeqID37 Reads | # SeqID38 Reads |
|---|---|---|
| # Reads @ 98% accuracy | 221 | 14,877 |
| # Reads @ 95% accuracy | 2,913 | 27,527 |
| # Reads @ 90% accuracy | 11,647 | 34,362 |
| # total reads | 24,291 | 40,578 |
| | % SeqID37 Reads | % SeqID38 Reads |
| % Reads @ 98% accuracy | 0.9% | 36.7% |
| % Reads @ 95% accuracy | 12.0% | 67.8% |
| % Reads @ 90% accuracy | 47.9% | 84.7% |
| % total reads | 100.0% | 100.0% |

Analysis of the Oligo 37 and Oligo 38 sequences showed that the control panel Oligos 37 and 38 comprise a high degree of secondary structure, which is known to cause errors in sequence determination. As such, the NGS output for these oligonucleotides was disregarded. While not being bound by theory and with an understanding that the theory is not required to practice the technology, it is contemplated that the high degree of secondary structure in Oligo 37 most likely explains its suppressed performance compared to Oligo 38. Consequently, it is contemplated that alternate designs may provide improved results for monitoring % GC sequencing performance monitoring between NGS systems or runs.

Similar experiments were conducted with Oligo 26 and Oligo 27. Table 7 shows the results of these experiments.

TABLE 7
Control Panel Oligo 26 (60% AT) & Oligo 27 (70% AT)

| | # SeqID26 Reads | # SeqID27 Reads |
|---|---|---|
| # Reads @ 98% accuracy | 42,616 | 23,750 |
| # Reads @ 95% accuracy | 51,929 | 26,881 |
| # Reads @ 90% accuracy | 53,940 | 27,655 |
| # total reads | 55,003 | 34,560 |
| | % SeqID26 Reads | % SeqID27 Reads |
| % Reads @ 98% accuracy | 77.5% | 68.7% |
| % Reads @ 95% accuracy | 94.4% | 77.8% |
| % Reads @ 90% accuracy | 98.1% | 80.0% |
| % total reads | 100.0% | 100.0% |

As expected, the % of mapped reads were lower for the higher % AT control panel Oligo 27 compared to Oligo 26.

In sum, the data collected during the development of embodiments of the technology provided herein indicate NGS control panel oligonucleotides included in NGS samples provide for monitoring the performance of different sequencing contexts alongside an NGS library. It is contemplated that the oligonucleotides of the NGS control panel find use to track the control panel's performance across multiple runs and/or NGS platforms and to correlate control panel performance to overall NGS run performance (e.g. ability to call variants of interest or ability to call variants with known challenging sequence content).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method for determining analytical sensitivity of a nucleic acid reaction comprising:
a. adding predetermined concentrations of a plurality of synthetic nucleic acids to a sample containing a target nucleic acid, wherein two or more different members of said plurality of synthetic nucleic acids differ from one another in concentration and/or sequence;
b. subjecting the mixture from (a) to a nucleic acid amplification procedure in which the synthetic nucleic acids and the target nucleic acid are amplified;
c. identifying the amplification products from (b) of the synthetic nucleic acids and target nucleic acid by identifying a measurable signal;
d. detecting the presence of or amount of target nucleic acid in the sample using the measurable signal from (c); and
e. determining the analytical sensitivity of the detection in (d) by analyzing the measurable signal generated by the synthetic nucleic acids.

2. The method of claim 1 wherein the nucleic acid reaction is a somatic mutation assay, a nucleic acid homopolymer assay, an AT-rich nucleic acid assay, a GC-rich nucleic acid assay, a short tandem repeat assay, a telomere repeat assay, a centromere repeat assay, a nucleic acid deletion assay, or a nucleic acid copy number assay.

3. The method of claim 1 wherein the identifying step comprises use of nucleic acid sequencing.

4. The method claim 1 wherein the identifying step comprises use of digital PCR.

5. The method of claim 1 wherein:
a. the nucleic acid reaction is a somatic mutation assay and the synthetic nucleic acids and the target nucleic acid differ by one or more single nucleotide substitution;
b. the nucleic acid reaction is a somatic mutation assay and the synthetic nucleic acids differ from each other by the location of the single nucleotide polymorphism;
c. the nucleic acid reaction is a somatic mutation assay and the synthetic nucleic acids contain each possible variation of the base at the location of the single nucleotide polymorphism;
d. the nucleic acid reaction is a nucleic acid homopolymer assay and the synthetic nucleic acids differ from each other and/or the target nucleic acid by homopolymer stretches of a single base repeated 2-25 times;
e. the nucleic acid reaction is a short tandem repeat assay and the synthetic nucleic acids differ from each other and/or the target nucleic acid by short tandem repeats;
f. the nucleic acid reaction is a GC-rich or AT-rich nucleic acid assay and the synthetic nucleic acids differ from each other and/or the target nucleic acid by % AT or % GC content;
g. the nucleic acid reaction is a centromere repeat assay or a telomere repeat assay and the synthetic nucleic acids differ from each other and/or the target nucleic acid by presence of, nature of, sequence context of, or number of telomeric, subtelomeric, or centromeric repeats; and/or
h. the nucleic acid reaction is a nucleic acid deletion assay and the synthetic nucleic acids differ from each other and/or the target nucleic acid by small nucleic acid deletions.

6. The method of claim 1, wherein the synthetic nucleic acids differ from each other and/or target nucleic acid by ribonucleic acid structures comprising one or more of the following: circles, pseudoknots, hairpins, self-complementary tails, single stranded pseudo circles, and transfer ribonucleic acid structures.

7. The method of claim 1 wherein the predetermined concentrations of synthetic nucleic acids differ from one another in molarity in ratios selected from the group consisting of: 1:1, 1:1.05, 1:10, 1:100, 1:1000, 1:10,000, 1:100,000, 1:1,000,000, and other ratios of the formula 1:10^{x} where x is a positive number.

8. The method of claim 7, wherein three or more different predetermined concentrations are used.

9. A kit for determining the specificity of a nucleic acid sequencing reaction comprising:
a. a plurality of synthetic nucleic acids in predetermined concentrations that differ in sequence and concentration from each other and that differ in sequence from a target nucleic acid;
b. nucleic acid amplification reagents comprising a plurality of primers that co-amplify said target nucleic acid and said plurality of synthetic nucleic acids; and
c. nucleic acid sequencing reagents.

10. The kit of claim 9, wherein
a. the synthetic nucleic acids differ from the target nucleic acid by a single nucleotide polymorphism;
b. the synthetic nucleic acids differ from each other by the location of the single nucleotide polymorphism;
c. the synthetic nucleic acids contain each possible variation of the base at the location of the single nucleotide polymorphism;
d. the synthetic nucleic acids differ from each other and/or the target nucleic acid by homopolymer stretches of a single base repeated 2-25 times;
e. the synthetic nucleic acids differ from each other and/or the target nucleic acid by short tandem repeats;
f. the synthetic nucleic acids differ from each other and/or the target nucleic acid by % GC content;
g. the synthetic nucleic acids differ from each other and/or the target nucleic acid by % AT content;
h. the synthetic nucleic acids differ from each other and/or the target nucleic acid sequence by telomeric, subtelomeric, or centromeric repeats; and/or
i. the synthetic nucleic acids differ from each other and/or the target nucleic acid by small nucleic acid deletions.

11. The kit of claim 9, wherein the synthetic nucleic acids differ from each other and/or the target nucleic acid by ribonucleic acid structures comprising one or more of the following: circles, pseudoknots, hairpins, self-complementary tails, single stranded pseudo circles, and transfer ribonucleic acid structures.

12. The kit of claim 9, wherein the predetermined concentrations of synthetic nucleic acids differ from one another in molarity in ratios selected from the group consisting of: 1:1.05, 1:10, 1:100, 1:1000, 1:10,000, 1:100,000, 1:1,000,000, and other ratios of the formula 1:10^{x} where x is a positive number.

13. A composition comprising:
a. a plurality of synthetic nucleic acids in predetermined concentrations that differ in sequence and concentration from each other and that differ in sequence from a target nucleic acid; and
b. nucleic acid amplification reagents comprising a plurality of primers that co-amplify said target nucleic acid and said plurality of synthetic nucleic acids.

14. The composition of claim 13 wherein the composition is a reaction mixture.

15. A composition comprising: a) amplicons generated from an amplification reaction employing the composition of claim 13; and b) sequencing reagents.

16. The composition of claim 15, wherein the composition is a reaction mixture.


SEQUENCE LISTING (178 sequences)

SEQ ID NO: 1 - DNA, 100 bp, Artificial Sequence
  Synthetic
acgttgcatactgacctaggtaagcgttgcgaatctggatatgcttaacccatggatcaattcgacgcgggttacgcctaaattggccagcgttagctaa

SEQ ID NO: 2 - DNA, 100 bp, Artificial Sequence
  Synthetic
acgttgcatgctgacctaggtaagcgttgcgaatctggatctgcttaacccatggatcacttcgacgcgggttacgcctaaattggcctgcgttagctaa

SEQ ID NO: 3 - DNA, 100 bp, Artificial Sequence
  Synthetic
acgttgcatcctgacctaggtaagcgttgcgaatctggatttgcttaacccatggatcatttcgacgcgggttacgcctaaattggcccgcgttagctaa

SEQ ID NO: 4 - DNA, 100 bp, Artificial Sequence
  Synthetic
acgttgcattctgacctaggtaagcgttgcgaatctggatgtgcttaacccatggatcagttcgacgcgggttacgcctaaattggccggcgttagctaa

SEQ ID NO: 5 - DNA, 100 bp, Artificial Sequence
  Synthetic
acgttgcatacagacctaggtaagcgttgcgaatctggacatgcttaacccatggatcaagtcgacgcgggttacgcctaaattggccagtgttagctaa

SEQ ID NO: 6 - DNA, 100 bp, Artificial Sequence
  Synthetic
acgttgcataccgacctaggtaagcgttgcgaatctggagatgcttaacccatggatcaactcgacgcgggttacgcctaaattggccagtgttagctaa

SEQ ID NO: 7 - DNA, 100 bp, Artificial Sequence; identical to SEQ ID NO: 1
  Synthetic

SEQ ID NO: 8 - DNA, 100 bp, Artificial Sequence
  Synthetic
acgttgcattctgacctaggtaagcgttgcgaatctggatatgcctaacccatggatcaattcgacgccggttacgcctaaattggccagcgttagctaa

SEQ ID NO: 9 - DNA, 100 bp, Artificial Sequence
  Synthetic
acgttgctttctgacctagggaagcgttgcgaaactggatatgcccaacccatggatcaaatcgacgcccgttacgcctaaattgggcagcgtttgctaa

SEQ ID NO: 10 - DNA, 100 bp, Artificial Sequence
  Synthetic
acgttgctttttgacctaggggagcgttgcgaaaatggatatgccccacccatggataaaatcgacgccccttacgcctaaatggggcagcgttttctaa

SEQ ID NO: 11 - DNA, 100 bp, Artificial Sequence
  Synthetic
acgttgctttttgacctagggggccgttgcgaaaaaggatatcccccacccatggataaaaacgacgccccctacgcctaaagggggcagctttttctaa

SEQ ID NO: 12 - DNA, 100 bp, Artificial Sequence
  Synthetic
acgttgttttttgacctaggggggcgttgcaaaaaaggatatccccccttcatggtaaaaaacgacgccccccacgcctaaggggggcagcttttttgaa

SEQ ID NO: 13 - DNA, 100 bp, Artificial Sequence
  Synthetic
acgttgtttttttacctagggggggattgcaaaaaaagatatccccccctcatggaaaaaaacgacgcccccccagcctagggggggcagctttttttaa

SEQ ID NO: 14 - DNA, 100 bp, Artificial Sequence
  Synthetic
acgttgttttttttcctaggggggggttgcaaaaaaaagtatccccccccgatggaaaaaaaagacgccccccccgcctggggggggcagttttttttaa

SEQ ID NO: 15 - DNA, 100 bp, Artificial Sequence
  Synthetic
acgtatttttttttcctagggggggggtgcaaaaaaaaatatcccccccccatggaaaaaaaaatgcccccccccgccgggggggggcatttttttttaa

SEQ ID NO: 16 - DNA, 100 bp, Artificial Sequence
  Synthetic
acgattttttttttcctggggggggggtgaaaaaaaaaatatcccccccccctgaaaaaaaaaatgccccccccccaaggggggggggatttttttttta

SEQ ID NO: 17 - DNA, 100 bp, Artificial Sequence
  Synthetic
acgtttttttttttccgggggggggggtgaaaaaaaaaaagccccccccccctaaaaaaaaaaatcccccccccccagggggggggggattttttttttt

SEQ ID NO: 18 - DNA, 100 bp, Artificial Sequence
  Synthetic
acttttttttttttcggggggggggggtaaaaaaaaaaaaccccccccccccaaaaaaaaaaaaccccccccccccggggggggggggtttttttttttt

SEQ ID NO: 19 - DNA, 106 bp, Artificial Sequence
  Synthetic
actttttttttttttgggggggggggggaaaaaaaaaaaaacccccccccccccaaaaaaaaaaaaacccccccccccccgggggggggggggttttttttttttt

SEQ ID NO: 20 - DNA, 100 bp, Artificial Sequence
  Synthetic
cgcggccggccggccggccggcgccggcgcgccggccgcgcgccgcggcggcggcgccgcccggcgcgcgggccgcggcccggccggcgcgcccgcgcgg

SEQ ID NO: 21 - DNA, 100 bp, Artificial Sequence
  Synthetic
cgcggccggacggccggcctgcgccggcgagccggccgctcgccgcggcagcggcgccgtccggcgcgcaggccgcggctcggccggcgagcccgcgcgt

SEQ ID NO: 22 - DNA, 100 bp, Artificial Sequence
  Synthetic
agcggccggatggccggcctacgccggcgatccggccgctagccgcggcatcggcgccgtacggcgcgcatgccgcggctaggccggcgatcccgcgcgt

SEQ ID NO: 23 - DNA, 100 bp, Artificial Sequence
  Synthetic
agcggccgaatggccggcttacgccggcaatccggccgttagccgcggaatcggcgccttacggcgcgaatgccgcggttaggccggcaatcccgcgctt

SEQ ID NO: 24 - DNA, 100 bp, Artificial Sequence
  Synthetic
aacggccgaattgccggcttaagccggcaattcggccgttaaccgcggaattggcgccttaaggcgcgaattccgcggttaagccggcaattccgcgctt

SEQ ID NO: 25 - DNA, 100 bp, Artificial Sequence
  Synthetic
aacggccaaattgccggtttaagccggaaattcggcctttaaccgcgaaattggcgctttaaggcgcaaattccgcgtttaagccggaaattccgcgttt

SEQ ID NO: 26 - DNA, 100 bp, Artificial Sequence
  Synthetic
aaaggccaaatttccggtttaaaccggaaatttggcctttaaacgcgaaatttgcgctttaaagcgcaaatttcgcgtttaaaccggaaatttcgcgttt

SEQ ID NO: 27 - DNA, 100 bp, Artificial Sequence
  Synthetic
aaaggcaaaatttccgttttaaaccgaaaatttggcttttaaacgcaaaatttgcgttttaaagcgaaaatttcgcttttaaaccgaaaatttcgctttt

SEQ ID NO: 28 - DNA, 100 bp, Artificial Sequence
  Synthetic
aaaagcaaaattttcgttttaaaacgaaaattttgcttttaaaagcaaaattttcgttttaaaacgaaaattttgcttttaaaacgaaaattttgctttt

SEQ ID NO: 29 - DNA, 100 bp, Artificial Sequence
  Synthetic
aaaagaaaaattttctttttaaaacaaaaattttgtttttaaaagaaaaattttctttttaaaacaaaaattttgtttttaaaacaaaaattttgttttt

SEQ ID NO: 30 - DNA, 100 bp, Artificial Sequence
  Synthetic
aaaaaaaaaattttttttttaaaaaaaaaattttttttttaaaaaaaaaattttttttttaaaaaaaaaattttttttttaaaaaaaaaatttttttttt

SEQ ID NO: 31 - DNA, 100 bp, Artificial Sequence
  Synthetic
aattataattaatatattattaaatataattaatatattattatataaatattatataattaaatattatatttatataaattatatatatattataata

SEQ ID NO: 32 - DNA, 100 bp, Artificial Sequence
  Synthetic
aattataatcaatatattagtaaatataactaatatattgttatataaacattatataagtaaatattacatttatatagattatatatctattataatg

SEQ ID NO: 33 - DNA, 100 bp, Artificial Sequence
  Synthetic
cattataatcgatatattagcaaatataacgaatatattgctatataaacgttatataagcaaatattacgtttatatagcttatatatcgattataatg

SEQ ID NO: 34 - DNA, 100 bp, Artificial Sequence
  Synthetic
cattataaccgatatattggcaaatataccgaatatatggctatataaccgttatataggcaaatattccgtttatatggcttatataccgattataagg

SEQ ID NO: 35 - DNA, 100 bp, Artificial Sequence
  Synthetic
ccttataaccggtatattggccaatataccggatatatggccatataaccggtatataggccaatattccggttatatggcctatataccggttataagg

SEQ ID NO: 36 - DNA, 100 bp, Artificial Sequence
  Synthetic
ccttatacccggtatatgggccaatatcccggatatagggccatatacccggtatatgggccaatatcccggttatagggcctatatcccggttataggg

SEQ ID NO: 37 - DNA, 100 bp, Artificial Sequence
  Synthetic
ccctatacccgggatatgggcccatatcccgggtatagggccctatacccgggatatgggcccatatcccgggtatagggcccatatcccgggtataggg

SEQ ID NO: 38 - DNA, 100 bp, Artificial Sequence
  Synthetic
ccctatccccgggataggggcccataccccgggtatggggccctatccccgggataggggcccataccccgggtatggggcccataccccgggtatgggg

SEQ ID NO: 39 - DNA, 100 bp, Artificial Sequence
  Synthetic
ccccatccccggggtaggggcccctaccccggggatggggccccatccccggggtaggggcccctaccccggggatggggcccctaccccggggatgggg

SEQ ID NO: 40 - DNA, 100 bp, Artificial Sequence
  Synthetic
ccccacccccggggtgggggcccctcccccggggagggggccccacccccggggtgggggcccctcccccggggagggggcccctcccccggggaggggg

SEQ ID NO: 41 - DNA, 100 bp, Artificial Sequence
  Synthetic
ccccccccccggggggggggccccccccccggggggggggccccccccccggggggggggccccccccccggggggggggccccccccccgggggggggg

SEQ ID NO: 42 - DNA, 200 bp, Artificial Sequence
  Synthetic
aagttgcataatgacctaggacagcgttgcagatctggattagcttaacctttggatcaatccgacgcggtgtacgcctaaattggccagcgttagctaacagttgcatactgacctaggccagcgttgccgatctggatgagcttaaccgttggatcaagccgacgcggggtacgcctaaattggccagcgttagctaa

SEQ ID NO: 43 - DNA, 200 bp, Artificial Sequence
  Synthetic
aaaatgcataatatcctaggacaccgttgcagagctggattatattaaccttttgatcaatctcacgcggtgtgcgcctaaattggccagcgttagctaacacatgcatactctcctaggcccccgttgccgcgctggatgagattaaccgtgtgatcaagcgcacgcggggggcgcctaaattggccagcgttagctaa

SEQ ID NO: 44 - DNA, 200 bp, Artificial Sequence
  Synthetic
aaaaaacataatatattaggacacacttgcagagagggattatataaacctttttttcaatctctcgcggtgtgtgcctaaattggccagcgttagctaacacacacatactctcttaggccccccttgccgcgcgggatgagagaaaccgtgtgttcaagcgcgcgcggggggggcctaaattggccagcgttagctaa

SEQ ID NO: 45 - DNA, 200 bp, Artificial Sequence
  Synthetic
aaaaaaaataatatatatggacacacacgcagagagagattatatataccttttttttaatctctctcggtgtgtgtgtaaattggccagcgttagctaacacacacatactctctctggccccccccgccgcgcgcgatgagagagaccgtgtgtgtaagcgcgcgcggggggggggtaaattggccagcgttagctaa

SEQ ID NO: 46 - DNA, 200 bp, Artificial Sequence
  Synthetic
aaaaaaaaaaatatatatatacacacacacagagagagagtatatatatatttttttttttctctctcggtgtgtgtgtgaattggccagcgttagctaacacacacacactctctctctcccccccccccgcgcgcgcggagagagagagtgtgtgtgtgcgcgcgcgcggggggggggaattggccagcgttagctaa

SEQ ID NO: 47 - DNA, 200 bp, Artificial Sequence
  Synthetic
aaattgcataaatacctaggaacgcgttgcaagtctggatacacttaaccactggatcaaacggacgcggaccacgcctaatatggccagatttagctaaatgttgcataatcacctaggagagcgttgcagttctggataggcttaaccagcggatcaagccgacgcggggtacgcctaaattggccagcgttagctaa

SEQ ID NO: 48 - DNA, 200 bp, Artificial Sequence
  Synthetic
taattgcatatatacctaggtacgcgttgctagtctggattcacttaacctctggatcaatcggacgcggtccacgcctattatggccagttttagctaattgttgcatattcacctaggtgagcgttgctgttctggattggcttaacctgcggatcaagccgacgcggggtacgcctaaattggccagcgttagctaa

SEQ ID NO: 49 - DNA, 200 bp, Artificial Sequence
  Synthetic
caattgcatacatacctaggcacgcgttgccagtctggatccacttaacccctggatcaaccggacgcggcccacgcctactatggccagctttagctaactgttgcatactcacctaggcgagcgttgccgttctggatcggcttaacccgcggatcaagccgacgcggggtacgcctaaattggccagcgttagctaa

SEQ ID NO: 50 - DNA, 200 bp, Artificial Sequence
  Synthetic
gaattgcatagatacctagggacgcgttgcgagtctggatgcacttaaccgctggatcaagcggacgcgggccacgcctagtatggccaggtttagctaagtgttgcatagtcacctaggggagcgttgcggttctggatgggcttaaccggcggatcaagccgacgcggggtacgcctaaattggccagcgttagctaa

SEQ ID NO: 51 - DNA, 200 bp, Artificial Sequence
  Synthetic
aaaaaacataaataattaggaacaacttgcaagaagggatacaacaaaccactacttcaaacgacggcggaccacccctaataataccagattattctaaatgatgcataatcatctaggagaagattgcagtagtggataggaggaaccagcagctcaagccgacgcggggtacgcctaaattggccagcgttagctaa

SEQ ID NO: 52 - DNA, 200 bp, Artificial Sequence
  Synthetic
taataacatatattattaggtactacttgctagtagggattcatcaaacctcttcttcaatcgtcggcggtcctcccctattattaccagttttttctaattgttgcatattcttctaggtgatgattgctgttgtggattggtggaacctgctgctcaagccgacgcggggtacgcctaaattggccagcgttagctaa

SEQ ID NO: 53 - DNA, 200 bp, Artificial Sequence
  Synthetic
caacaacatacatcattaggcaccacttgccagcagggatccaccaaacccctccttcaaccgccggcggcccccccctactactaccagcttcttctaactgctgcatactcctctaggcgacgattgccgtcgtggatcggcggaacccgccgctcaagccgacgcggggtacgcctaaattggccagcgttagctaa

SEQ ID NO: 54 - DNA, 200 bp, Artificial Sequence
  Synthetic
gaagaacatagatgattagggacgacttgcgaggagggatgcagcaaaccgctgcttcaagcggcggcgggccgcccctagtagtaccaggttgttctaagtggtgcatagtcgtctaggggaggattgcggtggtggatggggggaaccggcggctcaagccgacgcggggtacgcctaaattggccagcgttagctaa

SEQ ID NO: 55 - DNA, 200 bp, Artificial Sequence
  Synthetic
aaaaaaaaaaaataataatgaacaacaaccaagaagaagtacaacaacacactactactaacgacgacggaccaccaccaataattatagattattattaatgatgatgaatcatcatcgagaagaagacagtagtagttaggaggaggcagcagcagcagccgacgcggggtacgcctaaattggccagcgttagctaa

SEQ ID NO: 56 - DNA, 200 bp, Artificial Sequence
  Synthetic
taataataaatattattatgtactactacctagtagtagttcatcatcactcttcttctatcgtcgtcggtcctcctccattattattagtttttttttattgttgttgattcttcttcgtgatgatgactgttgttgtttggtggtggctgctgctgcagccgacgcggggtacgcctaaattggccagcgttagctaa

SEQ ID NO: 57 - DNA, 200 bp, Artificial Sequence
  Synthetic
caacaacaaacatcatcatgcaccaccacccagcagcagtccaccaccaccctcctccaaccgccgccggcccccccccactactactagcttcttcttactgctgctgactcctcctcgcgacgacgaccgtcgtcgttcggcggcggccgccgccgcagccgacgcggggtacgcctaaattggccagcgttagctaa

SEQ ID NO: 58 - DNA, 200 bp, Artificial Sequence
  Synthetic
gaagaagaaagatgatgatggacgacgaccgaggaggagtgcagcagcacgctgctgctagcggcggcgggccgccgccagtagtagtaggttgttgttagtggtggtgagtcgtcgtcgggaggaggacggtggtggttgggggggggcggcggcggcagccgacgcggggtacgcctaaattggccagcgttagctaa

SEQ ID NO: 59 - DNA, 100 bp, Artificial Sequence
  Synthetic
acgttgcatactgacctaggtaagcgttgcgaatctggatatgcttaacccatggatcaattcgacgcgggttacgcctaaattggccagcgcgttaggg

SEQ ID NO: 60 - DNA, 99 bp, Artificial Sequence
  Synthetic
acgttgcatactgacctaggtaagcgttgcgaatctggatatgcttaacccatggatcaattcgacgcgggttacgcctaaattggccttaggttaggg

SEQ ID NO: 61 - DNA, 99 bp, Artificial Sequence
  Synthetic
acgttgcatactgacctaggtaagcgttgcgaatctggatatgcttaacccatggatcaattcgacgcgggttacgcctaaattagggttaggttaggg

SEQ ID NO: 62 - DNA, 99 bp, Artificial Sequence
  Synthetic
acgttgcatactgacctaggtaagcgttgcgaatctggatatgcttaacccatggatcaattcgacgcgggttacgttagggttagggttaggttaggg

SEQ ID NO: 63 - DNA, 99 bp, Artificial Sequence
  Synthetic
acgttgcatactgacctaggtaagcgttgcgaatctggatatgcttaacccatggatcaattcgacgcggttagggttagggttagggttaggttaggg

SEQ ID NO: 64 - DNA, 99 bp, Artificial Sequence
  Synthetic
acgttgcatactgacctaggtaagcgttgcgaatctggatatgcttaacccatggatcaattcgttagggttagggttagggttagggttaggttaggg

SEQ ID NO: 65 - DNA, 99 bp, Artificial Sequence
  Synthetic
acgttgcatactgacctaggtaagcgttgcgaatctggatatgcttaacccatggatcttagggttagggttagggttagggttagggttaggttaggg

SEQ ID NO: 66 - DNA, 99 bp, Artificial Sequence
  Synthetic
acgttgcatactgacctaggtaagcgttgcgaatctggatatgcttaacccattagggttagggttagggttagggttagggttagggttaggttaggg

SEQ ID NO: 67 - DNA, 99 bp, Artificial Sequence
  Synthetic
acgttgcatactgacctaggtaagcgttgcgaatctggatatgcttttagggttagggttagggttagggttagggttagggttagggttaggttaggg

SEQ ID NO: 68 - DNA, 99 bp, Artificial Sequence
  Synthetic
acgttgcatactgacctaggtaagcgttgcgaatctggatttagggttagggttagggttagggttagggttagggttagggttagggttaggttaggg

SEQ ID NO: 69 - DNA, 99 bp, Artificial Sequence
  Synthetic
acgttgcatactgacctaggtaagcgttgcgaatttagggttagggttagggttagggttagggttagggttagggttagggttagggttaggttaggg

SEQ ID NO: 70 - DNA, 99 bp, Artificial Sequence
  Synthetic
acgttgcatactgacctaggtaagcgttttagggttagggttagggttagggttagggttagggttagggttagggttagggttagggttaggttaggg

SEQ ID NO: 71 - DNA, 99 bp, Artificial Sequence
  Synthetic
acgttgcatactgacctaggtattagggttagggttagggttagggttagggttagggttagggttagggttagggttagggttagggttaggttaggg

SEQ ID NO: 72 - DNA, 99 bp, Artificial Sequence
  Synthetic
acgttgcatactgaccttagggttagggttagggttagggttagggttagggttagggttagggttagggttagggttagggttagggttaggttaggg

SEQ ID NO: 73 - DNA, 99 bp, Artificial Sequence
  Synthetic
acgttgcatattagggttagggttagggttagggttagggttagggttagggttagggttagggttagggttagggttagggttagggttaggttaggg

SEQ ID NO: 74 - DNA, 99 bp, Artificial Sequence
  Synthetic
acgtttagggttagggttagggttagggttagggttagggttagggttagggttagggttagggttagggttagggttagggttagggttaggttaggg

SEQ ID NO: 75 - DNA, 101 bp, Artificial Sequence
  Synthetic
ttagggttagggttagggttagggttagggttagggttagggttagggttagggttagggttagggttagggttagggttagggttagggttaggttaggg

SEQ ID NO: 76 - DNA, 107 bp, Artificial Sequence
  Synthetic
ttagggttagggttagggttagggttagggttagggttagggttagggttagggttagggttagggttagggttagggttagggttagggttagggttaggttaggg

SEQ ID NO: 77 - DNA, 100 bp, Artificial Sequence
  Synthetic
acgttgcatactgacctaggtaagcgttgcgaatctggatatgcttaacccatggatcaattcgacgcgggttacgcctaaattggccagcgcgccctaa

SEQ ID NO: 78 - DNA, 100 bp, Artificial Sequence
  Synthetic
acgttgcatactgacctaggtaagcgttgcgaatctggatatgcttaacccatggatcaattcgacgcgggttacgcctaaattggccccctaaccctaa

SEQ ID NO: 79 - DNA, 100 bp, Artificial Sequence
  Synthetic
acgttgcatactgacctaggtaagcgttgcgaatctggatatgcttaacccatggatcaattcgacgcgggttacgcctaaaccctaaccctaaccctaa

SEQ ID NO: 80 - DNA, 100 bp, Artificial Sequence
  Synthetic
acgttgcatactgacctaggtaagcgttgcgaatctggatatgcttaacccatggatcaattcgacgcgggttacgccctaaccctaaccctaaccctaa

SEQ ID NO: 81 - DNA, 100 bp, Artificial Sequence
  Synthetic
acgttgcatactgacctaggtaagcgttgcgaatctggatatgcttaacccatggatcaattcgacgcggccctaaccctaaccctaaccctaaccctaa

SEQ ID NO: 82 - DNA, 100 bp, Artificial Sequence
  Synthetic
acgttgcatactgacctaggtaagcgttgcgaatctggatatgcttaacccatggatcaattcgccctaaccctaaccctaaccctaaccctaaccctaa

SEQ ID NO: 83 - DNA, 100 bp, Artificial Sequence
  Synthetic
acgttgcatactgacctaggtaagcgttgcgaatctggatatgcttaacccatggatcccctaaccctaaccctaaccctaaccctaaccctaaccctaa

SEQ ID NO: 84 - DNA, 100 bp, Artificial Sequence
  Synthetic
acgttgcatactgacctaggtaagcgttgcgaatctggatatgcttaacccaccctaaccctaaccctaaccctaaccctaaccctaaccctaaccctaa

SEQ ID NO: 85 - DNA, 100 bp, Artificial Sequence
  Synthetic
acgttgcatactgacctaggtaagcgttgcgaatctggatatgcttccctaaccctaaccctaaccctaaccctaaccctaaccctaaccctaaccctaa

SEQ ID NO: 86 - DNA, 100 bp, Artificial Sequence; identical to SEQ ID NO: 85
  Synthetic

SEQ ID NO: 87 - DNA, 100 bp, Artificial Sequence
  Synthetic
acgttgcatactgacctaggtaagcgttccctaattagggttagggttagaaccctaaccctaaccctaaccctaaccctaaccctaaccctaaccctaa

SEQ ID NO: 88 - DNA, 100 bp, Artificial Sequence
  Synthetic
acgttgcatactgacctaggtaccctaaccctaattagggttagggttagaaccctaaccctaaccctaaccctaaccctaaccctaaccctaaccctaa

SEQ ID NO: 89 - DNA, 100 bp, Artificial Sequence
  Synthetic
acgttgcatactgaccccctaaccctaaccctaattagggttagggttagaaccctaaccctaaccctaaccctaaccctaaccctaaccctaaccctaa

SEQ ID NO: 90 - DNA, 100 bp, Artificial Sequence; identical to SEQ ID NO: 89
  Synthetic

SEQ ID NO: 91 - DNA, 100 bp, Artificial Sequence
  Synthetic
acgttgcataccctaaccctaaccctaaccctaattagggttagggttagaaccctaaccctaaccctaaccctaaccctaaccctaaccctaaccctaa

SEQ ID NO: 92 - DNA, 100 bp, Artificial Sequence
  Synthetic
acgtccctaaccctaaccctaaccctaaccctaattagggttagggttagaaccctaaccctaaccctaaccctaaccctaaccctaaccctaaccctaa

SEQ ID NO: 93 - DNA, 102 bp, Artificial Sequence
  Synthetic
ccctaaccctaaccctaaccctaaccctaaccctaattagggttagggttagaaccctaaccctaaccctaaccctaaccctaaccctaaccctaaccctaa

SEQ ID NO: 94 - DNA, 108 bp, Artificial Sequence
  Synthetic
ccctaaccctaaccctaaccctaaccctaaccctaaccctaattagggttagggttagaaccctaaccctaaccctaaccctaaccctaaccctaaccctaaccctaa

SEQ ID NO: 95 - DNA, 6 bp, Unknown
  Vertebrates (e.g., Human, mouse, Xenopus)
ttaggg

SEQ ID NO: 96 - DNA, 6 bp, Neurospora crassa
ttaggg

SEQ ID NO: 97 - DNA, 6 bp, Unknown
  Slime moulds (e.g., Physarum, Didymium)
ttaggg

SEQ ID NO: 98 - DNA, 9 bp, Unknown
  Slime moulds (e.g., Dictyostelium)
agggggggg

SEQ ID NO: 99 - DNA, 6 bp, Unknown
  Kinetoplastid protozoa (e.g., Trypanosoma, Crithidia)
ttaggg

SEQ ID NO: 100 - DNA, 6 bp, Unknown
  Ciliate protozoa (e.g., Tetrahymena, Glaucoma)
ttgggg

SEQ ID NO: 101 - DNA, 6 bp, Unknown
  Ciliate protozoa (e.g., Paramecium)
ttgggn

SEQ ID NO: 102 - DNA, 8 bp, Unknown
  Ciliate protozoa (e.g., Oxytricha, Stylonychia, Euplotes)
ttttgggg

SEQ ID NO: 103 - DNA, 7 bp, Unknown
  Apicomplexan protozoa (e.g., Plasmodium)
ttagggn

SEQ ID NO: 104 - DNA, 7 bp, Arabidopsis thaliana
tttaggg

SEQ ID NO: 105 - DNA, 8 bp, Unknown
  Green algae (e.g., Chlamydomonas)
ttttaggg

SEQ ID NO: 106 - DNA, 5 bp, Bombyx mori
ttagg

SEQ ID NO: 107 - DNA, 6 bp, Ascaris lumbricoides
ttaggc

SEQ ID NO: 108 - DNA, 14 bp, Schizosaccharomyces pombe
  misc_feature (5)..(5): this nucleotide may be absent
ttacacgggggggg

SEQ ID NO: 109 - DNA, 13 bp, Saccharomyces cerevisiae
tgtgggtgtggtg

SEQ ID NO: 110 - DNA, 16 bp, Saccharomyces cerevisiae
  misc_feature (3)..(3): this nucleotide may be absent
gggtgtgtgtgtgtgt

SEQ ID NO: 111 - DNA, 8 bp, Saccharomyces castellii
tctgggtg

SEQ ID NO: 112 - DNA, 15 bp, Candida glabrata
ggggtctgggtgctg

SEQ ID NO: 113 - DNA, 23 bp, Candida albicans
ggtgtacggatgtctaacttctt

SEQ ID NO: 114 - DNA, 23 bp, Candida tropicalis
  misc_feature (7)..(7): N = C or A
ggtgtanggatgtcacgatcatt

SEQ ID NO: 115 - DNA, 23 bp, Candida maltosa
ggtgtacggatgcagactcgctt

SEQ ID NO: 116 - DNA, 7 bp, Unknown
  Budding yeasts (e.g., Candida guillermondii)
ggtgtac

SEQ ID NO: 117 - DNA, 25 bp, Candida pseudotropicalis
ggtgtacggatttgattagttatgt

SEQ ID NO: 118 - DNA, 25 bp, Kluyveromyces lactis
ggtgtacggatttgattaggtatgt

SEQ ID NO: 119 - DNA, 100 bp, Artificial Sequence
  Synthetic
acgttgcatactgacctaggtaagcgttgcgaatctggatatgcttaacccatggatcaattcgacgcgggttacgcctaaattggccagcgcgctggaa

SEQ ID NO: 120 - DNA, 100 bp, Artificial Sequence
  Synthetic
acgttgcatactgacctaggtaagcgttgcgaatctggatatgcttaacccatggatcaattcgacgcgggttacgcctaaattggccagtggaatggaa

SEQ ID NO: 121 - DNA, 100 bp, Artificial Sequence
  Synthetic
acgttgcatactgacctaggtaagcgttgcgaatctggatatgcttaacccatggatcaattcgacgcgggttacgcctaaattgtggaatggaatggaa

SEQ ID NO: 122 - DNA, 100 bp, Artificial Sequence
  Synthetic
acgttgcatactgacctaggtaagcgttgcgaatctggatatgcttaacccatggatcaattcgacgcgggttacgcctatggaatggaatggaatggaa

SEQ ID NO: 123 - DNA, 100 bp, Artificial Sequence
  Synthetic
acgttgcatactgacctaggtaagcgttgcgaatctggatatgcttaacccatggatcaattcgacgcgggttactggaatggaatggaatggaatggaa

SEQ ID NO: 124 - DNA, 100 bp, Artificial Sequence
  Synthetic
acgttgcatactgacctaggtaagcgttgcgaatctggatatgcttaacccatggatcaattcgacgcggtggaatggaatggaatggaatggaatggaa

SEQ ID NO: 125 - DNA, 100 bp, Artificial Sequence
  Synthetic
acgttgcatactgacctaggtaagcgttgcgaatctggatatgcttaacccatggatcaattcgatggaatggaatggaatggaatggaatggaatggaa

SEQ ID NO: 126 - DNA, 100 bp, Artificial Sequence
  Synthetic
acgttgcatactgacctaggtaagcgttgcgaatctggatatgcttaacccatggatcaatggaatggaatggaatggaatggaatggaatggaatggaa

SEQ ID NO: 127 - DNA, 100 bp, Artificial Sequence
  Synthetic
acgttgcatactgacctaggtaagcgttgcgaatctggatatgcttaacccatggtggaatggaatggaatggaatggaatggaatggaatggaatggaa

SEQ ID NO: 128 - DNA, 100 bp, Artificial Sequence
  Synthetic
acgttgcatactgacctaggtaagcgttgcgaatctggatatgcttaacctggaatggaatggaatggaatggaatggaatggaatggaatggaatggaa

SEQ ID NO: 129 - DNA, 100 bp, Artificial Sequence
  Synthetic
acgttgcatactgacctaggtaagcgttgcgaatctggatatgcttggaatggaatggaatggaatggaatggaatggaatggaatggaatggaatggaa

SEQ ID NO: 130 - DNA, 100 bp, Artificial Sequence
  Synthetic
acgttgcatactgacctaggtaagcgttgcgaatctggattggaatggaatggaatggaatggaatggaatggaatggaatggaatggaatggaatggaa

SEQ ID NO: 131 - DNA, 100 bp, Artificial Sequence
  Synthetic
acgttgcatactgacctaggtaagcgttgcgaatctggaatggaatggaatggaatggaatggaatggaatggaatggaatggaatggaatggaatggaa

SEQ ID NO: 132 - DNA, 100 bp, Artificial Sequence
  Synthetic
acgttgcatactgacctaggtaagcgttgctggaatggaatggaatggaatggaatggaatggaatggaatggaatggaatggaatggaatggaatggaa

SEQ ID NO: 133 - DNA, 100 bp, Artificial Sequence
  Synthetic
acgttgcatactgacctaggtaagctggaatggaatggaatggaatggaatggaatggaatggaatggaatggaatggaatggaatggaatggaatggaa

SEQ ID NO: 134 - DNA, 100 bp, Artificial Sequence
  Synthetic
acgttgcatactgacctaggtggaatggaatggaatggaatggaatggaatggaatggaatggaatggaatggaatggaatggaatggaatggaatggaa

SEQ ID NO: 135 - DNA, 100 bp, Artificial Sequence
  Synthetic
acgttgcatactgactggaatggaatggaatggaatggaatggaatggaatggaatggaatggaatggaatggaatggaatggaatggaatggaatggaa

SEQ ID NO: 136 - DNA, 100 bp, Artificial Sequence
  Synthetic
acgttgcatatggaatggaatggaatggaatggaatggaatggaatggaatggaatggaatggaatggaatggaatggaatggaatggaatggaatggaa

SEQ ID NO: 137 - DNA, 100 bp, Artificial Sequence
  Synthetic
acgtttggaatggaatggaatggaatggaatggaatggaatggaatggaatggaatggaatggaatggaatggaatggaatggaatggaatggaatggaa

SEQ ID NO: 138 - DNA, 100 bp, Artificial Sequence
  Synthetic
tggaatggaatggaatggaatggaatggaatggaatggaatggaatggaatggaatggaatggaatggaatggaatggaatggaatggaatggaatggaa

SEQ ID NO: 139 - DNA, 100 bp, Artificial Sequence
  Synthetic
acgttgcatactgacctaggtaagcgttgcgaatctggatatgcttaacccatggatcaattcgacgcgggttacgcctaaattggccagcgcgcttcca

SEQ ID NO: 140 - DNA, 100 bp, Artificial Sequence
  Synthetic
acgttgcatactgacctaggtaagcgttgcgaatctggatatgcttaacccatggatcaattcgacgcgggttacgcctaaattggccagttccattcca

SEQ ID NO: 141 - DNA, 100 bp, Artificial Sequence
  Synthetic
acgttgcatactgacctaggtaagcgttgcgaatctggatatgcttaacccatggatcaattcgacgcgggttacgcctaaattgttccattccattcca

SEQ ID NO: 142 - DNA, 100 bp, Artificial Sequence
  Synthetic
acgttgcatactgacctaggtaagcgttgcgaatctggatatgcttaacccatggatcaattcgacgcgggttacgcctattccattccattccattcca

SEQ ID NO: 143 - DNA, 100 bp, Artificial Sequence
  Synthetic
acgttgcatactgacctaggtaagcgttgcgaatctggatatgcttaacccatggatcaattcgacgcgggttacttccattccattccattccattcca

SEQ ID NO: 144 - DNA, 100 bp, Artificial Sequence
  Synthetic
acgttgcatactgacctaggtaagcgttgcgaatctggatatgcttaacccatggatcaattcgacgcggttccattccattccattccattccattcca

SEQ ID NO: 145 - DNA, 100 bp, Artificial Sequence
  Synthetic
acgttgcatactgacctaggtaagcgttgcgaatctggatatgcttaacccatggatcaattcgattccattccattccattccattccattccattcca

SEQ ID NO: 146 - DNA, 100 bp, Artificial Sequence
  Synthetic
acgttgcatactgacctaggtaagcgttgcgaatctggatatgcttaacccatggatcaattccattccattccattccattccattccattccattcca

SEQ ID NO: 147 - DNA, 100 bp, Artificial Sequence
  Synthetic
acgttgcatactgacctaggtaagcgttgcgaatctggatatgcttaacccatggttccattccattccattccattccattccattccattccattcca

SEQ ID NO: 148 - DNA, 100 bp, Artificial Sequence
  Synthetic
acgttgcatactgacctaggtaagcgttgcgaatctggatatgcttaaccttccattccattccattccattccattccattccattccattccattcca

SEQ ID NO: 149 - DNA, 100 bp, Artificial Sequence
  Synthetic
acgttgcatactgacctaggtaagcgttgcgaatctggatatgctttccattccattccattccattccattccattccattccattccattccattcca

SEQ ID NO: 150 - DNA, 100 bp, Artificial Sequence
  Synthetic
acgttgcatactgacctaggtaagcgttgcgaatctggatttccattccattccattccattccattccattccattccattccattccattccattcca

SEQ ID NO: 151 - DNA, 100 bp, Artificial Sequence
  Synthetic
acgttgcatactgacctaggtaagcgttgcgaatcttccattccattccattccattccattccattccattccattccattccattccattccattcca

SEQ ID NO: 152 - DNA, 100 bp, Artificial Sequence
  Synthetic
acgttgcatactgacctaggtaagcgttgcttccattccattccattccattccattccattccattccattccattccattccattccattccattcca

SEQ ID NO: 153 - DNA, 100 bp, Artificial Sequence
  Synthetic
acgttgcatactgacctaggtaagcttccattccattccattccattccattccattccattccattccattccattccattccattccattccattcca

SEQ ID NO: 154 - DNA, 100 bp, Artificial Sequence
  Synthetic
acgttgcatactgacctaggttccattccattccattccattccattccattccattccattccattccattccattccattccattccattccattcca

SEQ ID NO: 155 - DNA, 100 bp, Artificial Sequence
  Synthetic
acgttgcatactgacttccattccattccattccattccattccattccattccattccattccattccattccattccattccattccattccattcca

SEQ ID NO: 156 - DNA, 100 bp, Artificial Sequence
  Synthetic
acgttgcatattccattccattccattccattccattccattccattccattccattccattccattccattccattccattccattccattccattcca

SEQ ID NO: 157 - DNA, 100 bp, Artificial Sequence
  Synthetic
acgttttccattccattccattccattccattccattccattccattccattccattccattccattccattccattccattccattccattccattcca

SEQ ID NO: 158 - DNA, 100 bp, Artificial Sequence
  Synthetic
ttccattccattccattccattccattccattccattccattccattccattccattccattccattccattccattccattccattccattccattcca

SEQ ID NO: 159 - DNA, 100 bp, Artificial Sequence
  Synthetic
tctgattcagctagtccagctaagcgttgcgaatctggatatgcttaacccatggatcaattcgacgcgggttacgcctaaattggccagcgttagctaa

SEQ ID NO: 160 - DNA, 100 bp, Artificial Sequence
  Synthetic
ctgtcggtatagcagaatcgtaagcgttgcgaatctggatatgcttaacccatggatcaattcgacgcgggttacgcctaaattggccagcgttagctaa

SEQ ID NO: 161 - DNA, 100 bp, Artificial Sequence
  Synthetic
agcatcaagctctgcatgcctaagcgttgcgaatctggatatgcttaacccatggatcaattcgacgcgggttacgcctaaattggccagcgttagctaa

SEQ ID NO: 162 - DNA, 100 bp, Artificial Sequence
  Synthetic
gatcgacactgatcagacagtaagcgttgcgaatctggatatgcttaacccatggatcaattcgacgcgggttacgcctaaattggccagcgttagctaa

SEQ ID NO: 163 - DNA, 100 bp, Artificial Sequence; identical to SEQ ID NO: 1
  Synthetic

SEQ ID NO: 164 - DNA, 100 bp, Artificial Sequence; identical to SEQ ID NO: 2
  Synthetic

SEQ ID NO: 165 - DNA, 100 bp, Artificial Sequence; identical to SEQ ID NO: 3
  Synthetic

SEQ ID NO: 166 - DNA, 100 bp, Artificial Sequence; identical to SEQ ID NO: 4
  Synthetic

SEQ ID NO: 167 - DNA, 100 bp, Artificial Sequence; identical to SEQ ID NO: 10
  Synthetic

SEQ ID NO: 168 - DNA, 100 bp, Artificial Sequence; identical to SEQ ID NO: 159
  Synthetic

SEQ ID NO: 169 - DNA, 100 bp, Artificial Sequence; identical to SEQ ID NO: 160
  Synthetic

SEQ ID NO: 170 - DNA, 100 bp, Artificial Sequence; identical to SEQ ID NO: 161
  Synthetic

SEQ ID NO: 171 - DNA, 100 bp, Artificial Sequence; identical to SEQ ID NO: 162
  Synthetic

SEQ ID NO: 172 - DNA, 100 bp, Artificial Sequence; identical to SEQ ID NO: 37
  Synthetic

SEQ ID NO: 173 - DNA, 100 bp, Artificial Sequence; identical to SEQ ID NO: 38
  Synthetic

SEQ ID NO: 174 - DNA, 100 bp, Artificial Sequence; identical to SEQ ID NO: 26
  Synthetic

SEQ ID NO: 175 - DNA, 100 bp, Artificial Sequence; identical to SEQ ID NO: 27
  Synthetic

SEQ ID NO: 176 - DNA, 43 bp, Artificial Sequence
  Synthetic
ccatctcatccctgcgtgtctccgactcagctaaggtaacgat

SEQ ID NO: 177 - DNA, 41 bp, Artificial Sequence
  Synthetic
atcaccgactgcccatagagaggaaagcggaggcgtagtgg

SEQ ID NO: 178 - DNA, 10 bp, Artificial Sequence
  Synthetic
ctaaggtaac